(19) United States
(12) Patent Application Publication
(10) Pub. No.: US 2021/0407657 A1
(43) Pub. Date: Dec. 30, 2021

(54) OFFICE ASSISTANT TOOL

(71) Applicant: Clover Health, Jersey, NJ (US)

(72) Inventors: Mark Spektor, New York, NY (US); Paul Zumbrun, San Francisco, CA (US); Clara Wu, San Francisco, CA (US); Kejia Zhu, San Francisco, CA (US); Ankit Patel, San Francisco, CA (US)

(21) Appl. No.: 16/912,483

(22) Filed: Jun. 25, 2020

(51) Int. Cl. G16H 40/20; G16H 10/60; G16H 15/00; G16H 50/20; G16H 20/10; G06Q 10/10; G16H 50/70; G06Q 30/04; G06Q 10/06; G16H 70/20

(52) U.S. Cl. CPC G16H 40/20; G16H 10/60; G16H 15/00; G16H 50/20; G16H 70/20; G06Q 10/1097; G16H 50/70; G06Q 30/04; G06Q 10/06316; G16H 20/10

(57) ABSTRACT

Techniques are described herein that provide an interface for consolidating medical care tasks to increase efficiency associated with managing a medical office. The medical care tasks may include scheduling appointments, providing clinical assessment data for an appointment, renewing medications, submitting insurance claims, reviewing payment data, and the like. A service provider may access member data to determine that a member is due for an appointment, such as that associated with a clinical visit, a procedure, a screening, a medical test, or the like. In some examples, the service provider may determine that a medication associated with the member is due for renewal or modification. The service provider may generate a medical care task associated with the appointment and/or medication renewal or modification and send the medical care task to an application on a computing device associated with a medical provider to address the medical care task.

### BACKGROUND

People generally visit medical providers for routine check-ups and procedures, and also for specific issues, such as illness, surgical follow-up, and the like. Each visit between a medical provider and a patient may include copious amounts of coordination outside of the visit, such as to schedule the visit, provide data associated with the patient to the medical provider, verify insurance for the patient, submit an insurance claim, and the like. Many current systems for managing a medical provider office separate the coordination tasks, thereby increasing a total time for an associate of the medical provider (e.g., office staff) to complete the coordination tasks.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is a schematic view of an example system usable to implement an office assistant tool, as described herein.

FIGS. 2A and 2B illustrate an example interface in which clinical assessments may be reviewed and generated. FIG. 2A illustrates an example interface in which previously generated clinical assessments may be reviewed. FIG. 2B illustrates the example interface configured for generating a new clinical assessment.

FIG. 3 illustrates an example interface associated with a care coordination task page.

FIG. 4 illustrates an example interface associated with a patient medication review.

FIG. 5 illustrates an example interface associated with a patient procedure review.

FIG. 6 illustrates an example interface associated with a clinical visit scheduling review.

FIG. 7 illustrates example interface associated with an insurance claim submission and payment review.

FIG. 8 illustrates a block diagram illustrating an example system of computing devices usable to implement example techniques described herein.

FIG. 9 illustrates an example process for providing a clinical assessment to a medical provider, utilizing the techniques described herein.

FIG. 10 illustrates an example process for determining that a member is due for a medication renewal and sending a notification of the medication renewal to a medical provider associated with the member, utilizing the techniques described herein.

FIG. 11 illustrates an example process for determining that a member is due for a procedure and sending a notification of the procedure to a medical provider associated with the member, utilizing the techniques described herein.

FIG. 12 illustrates an example process for providing a financial report to a medical provider, utilizing the techniques described herein.

### DETAILED DESCRIPTION

This application describes techniques for consolidating coordination tasks and increasing an efficiency associated with managing a medical office. In some instances, the coordination tasks may be consolidated on an application managed by a service provider. The application may enable increased communication and information flow between the office staff and the service provider, thereby greatly enhancing a service of care provided to members associated with the service provider.

An associate of a medical provider (e.g., office staff, assistant, associate, etc.) may determine that a patient (e.g., member) associated with the service provider is scheduled for an appointment (e.g., clinical visit). The associate may access an instance of the application to request a clinical assessment (e.g., an interface associated with a clinical visit, visit form, etc.). The request may include identifying information associated with the member (e.g., name, identifier, date of birth, etc.) and/or information associated with the appointment (e.g., provider, location, date, time, etc.). The associate may submit the request to the service provider for processing. The service provider may be configured to provide one or more services to the member and/or the medical provider, such as insurance services, clinical visit assistance services, referral services, scheduling services, and the like. The service provider may receive the information and generate the interface to assist a medical provider during the clinical visit.

In various example, the service provider may generate the clinical assessment based on member data, such as that stored in a member record associated with the member. The member may include a member associated with the service provider. The member data may include demographic information, medical history (e.g., previous diagnoses, medical procedures, surgeries, etc.), laboratory results (e.g., glucose, cholesterol, etc.), medical test results (e.g., Echo stress test result, EKG, etc.), member location information (e.g., home address, work address, etc.), pharmacological information (e.g., prescriptions, prescription fill information (e.g., last fill, expirations, etc.), preferred pharmacy, etc.). In some examples, the service provider may access the member data to determine information to include in the clinical assessment associated with the clinical visit between the medical provider and the member. In such examples, the clinical assessment may be tailored to an individual member at a particular time.

In various examples, the clinical assessment may include one or more or a potential diagnosis for the member (e.g., undiagnosed conditions, unconfirmed diagnoses), medication data, a gap in care (e.g., screening, procedure, test, surgery, consultation, etc. due for the member), and/or clinical recommendations associated with the member. In some examples, the clinical assessment may provide a means by which the medical provider may submit a referral for a procedure to the service provider, such as for approval.

In various examples, the service provider may send the clinical assessment to the medical provider via the instance of the application. In some examples, the service provider may cause an indication of the clinical assessment to surface or otherwise be presented on a main page associated with the application. In various examples, the main page may include an interface via which one or more clinical assessments may be displayed, reviewed, and/or submitted. The one or more clinical assessments displayed on the main page may include those that are created (e.g., clinical visit pending), signed and awaiting submission, submitted, and/or awaiting correction. In some examples, the main page may provide the office staff with a quick reference of clinical assessment tasks to be completed, such as submitting and/or correcting a clinical assessment.

In various examples, the office staff may determine one or more care coordination tasks by accessing a care coordination task page associated with the application. In some examples, the service provider may determine one or more care coordination tasks associated with one or more members. The care coordination task may include any task related to the health care of the one or more members, such as a medication renewal, a procedure that is due (e.g., gap in care), a clinical visit due to be scheduled, and the like. For example, the office staff may receive, via the care coordination task page, an indication that a member is due for a clinical visit. Based at least in part on the indication, the office staff may schedule the clinical visit and submit a request for a clinical assessment, such as that described above.

In some examples, the office staff may select a task via the care coordination task page. Responsive to receiving an indication of selection of the task, the service provider may cause data associated with the task to surface on the application. The data may include relevant information about the task, such as to adequately inform the office staff and/or medical provider about a service to provide to the member. For example, the task may include a medication renewal and the relevant information may include prescription information, such as a name of a drug, a length of fill, last fill date, expiration date, preferred pharmacy, prescriber, and the like.

In some examples, the application may provide a means by which the office staff may address the care coordination task. In some examples, the office staff may provide the service provider with updated information regarding the care coordination task. The updated information may include data associated with renewed medications, appointments scheduled, and the like. Responsive to receiving the updated information, the service provider may update member data.

In some examples, the service provider may access the member data to determine that a member has a care coordination task that is outstanding. For example, the service provider may determine that a member is due for a clinical visit within a threshold time. In various examples, responsive to determining that the member has an outstanding care coordination task, the service provider may cause a notification thereof to surface on a display of a computing device associated with a medical provider (e.g., office staff computer). The notification may provide an indication of a new care coordination task that is due. In some examples, responsive to receiving the notification, the office staff may access the data associated with the task to address (e.g., review, complete, send to the medical provider, etc.) the task. Continuing the example from above, service provider may surface an indication that the member is due for the clinical visit. Responsive to receiving the indication, the office staff may schedule an appointment for the clinical visit.

In various examples, the application may provide a means by which the office staff may review financial data associated with the service provider. The financial data may include one or more payments from the service provider (e.g., insurance company) to the medical provider, such as based on claims submitted for services rendered. In some examples, the office staff may access the financial data on a payments page of the application. In some examples, the office staff may request, via the payments page, a financial report of one or more payments from the service provider to the medical provider. In such examples, responsive to receiving the request, the service provider may provide the financial report with the one or more payments.

The techniques described herein improve a user interface of a computing device by providing real-time and/or near real-time information about a patient to office staff associated with a medical provider. The information provided via the interface may not otherwise be readily available to the office staff, such as not without a comprehensive review of a medical record associated with the patient. The service provider's unique position in the medical industry and relationship with the medical provider and the patient enables the service provider to provide services to the medical provider (and benefiting the patient) that would not otherwise be available. The services may include generating clinical assessments and other care coordination tasks, such as scheduling appointments, medication renewals, and the like. The services provided due to the service provider's unique position may enable a medical provider to provide a substantially improved level of care to the patient.

Although primarily discussed herein as an application configured to surface multiple interfaces associated with care coordination tasks to an associate of a medical provider (e.g., office staff), any or all of the interfaces and/or care coordination tasks discussed herein may be performed by a medical provider alone, such as in a single provider office.

These and other aspects are described further below with reference to the accompanying drawings. The drawings are merely example implementations and should not be construed to limit the scope of the claims. For example, while examples are illustrated in the context of a user interface for a mobile device, the techniques may be implemented using any computing device and the user interface may be adapted to the size, shape, and configuration of the particular computing device. Also, while many of the examples are given in the context of providing medical services, the techniques described herein may also be applied to any other context associated with managing care, such as record reviews, scheduling appointments, and the like.

### Example System Architecture

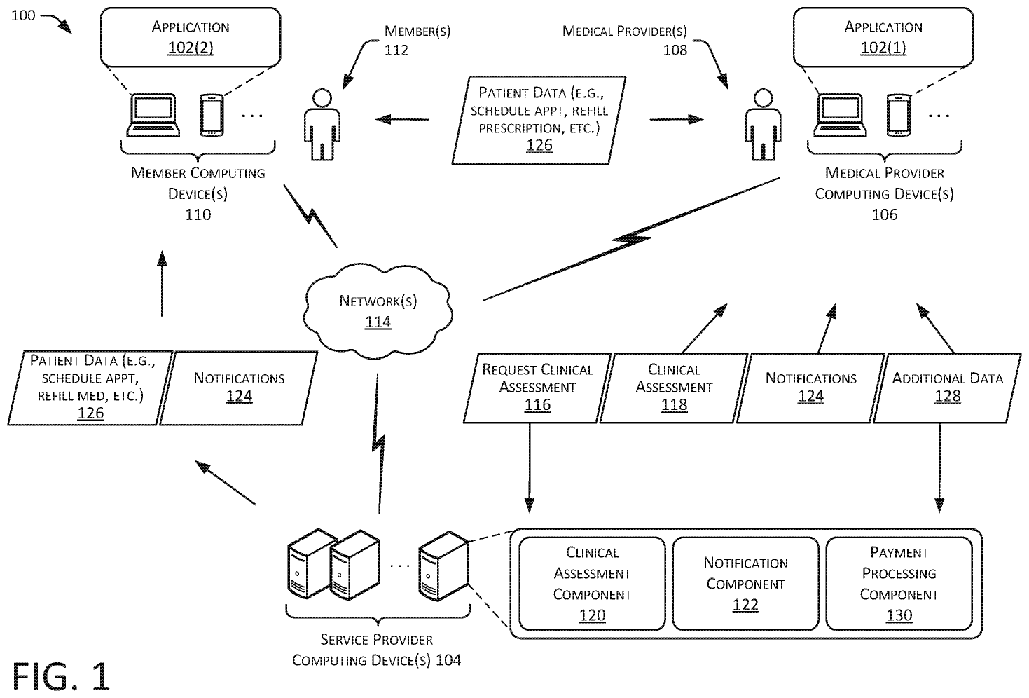

FIG. 1 is a schematic view of an example system 100 usable to implement the techniques described herein to provide relevant information via an instance of an application 102 via the system 100. In some examples, the system 100 may include a one or more service provider computing devices 104 (e.g., service provider 104) configured to manage the application 102, such as to provide the relevant information to one or more medical provider computing devices 106 (e.g., provider device(s) 106) associated with one or more medical providers 108. In various examples, the service provider computing device(s) 104 may additionally configured to provide information to one or more member computing devices 110 associated with one or more members 112. In various examples, the provider device(s) 106 may include a first instance of the application 102(1) and the member device(s) 110 may include a second instance of the application 102(2), to facilitate information flow to the medical provider(s) 108 and the member(s) 112.

Each of the provider device(s) 106 and the member device(s) 110 include one or more processors and memory storing computer executable instructions to implement the functionality discussed herein attributable to the respective computing devices. In some examples, the provider device(s) 106 and the member device(s) 110 may include desktop computers, laptop computers, tablet computers, mobile devices (e.g., smart phones or other cellular or mobile phones, mobile gaming devices, portable media devices, etc.), or other suitable computing devices. The provider device(s) 106 and the member device(s) 110 may execute one or more client applications, such as a web browser (e.g., Microsoft Windows Internet Explorer, Mozilla Firefox, Apple Safari, Google Chrome, Opera, etc.) or a native or special-purpose client application (e.g., service provider application, etc.), to access and view information provided by the service provider computing device(s) 104 over network 114.

In various examples, the provider device(s) 106 may include a single computing device. For example, a small medical office may include a single computing device for managing patient services, such as to schedule a clinical visit, prepare for a clinical visit, renew a medication, submit an insurance claim, and the like. In some examples, the provider device(s) 106 may include one or more other computing devices, any or all of which may include one or more processors and memory storing computer executable instructions to implement the functionality described herein. For example, a larger medical office may include a first set of computing devices associated with medical office staff, such as schedule clinical visits, prepare for clinical visits, submit insurance claims, and the like, and a second set of computing devices associated with the medical provider, such as for conducting clinical visits, renewing medications, and the like.

In some examples, the first instance of the application 102(1) may include one or more APIs configured to provide the medical provider(s) 108 functionalities within the first instance of the application 102(1) that differ from the second instance of the application 102(2). In some examples, the API(s) may include an enterprise client that enables multiple agents associated with the medical provider(s) 108 to access and respond to requests for information from the service provider computing device(s) 104 over the network 114.

Network 114 may represent a network or collection of networks (such as the Internet, a corporate intranet, a virtual private network (VPN), a local area network (LAN), a wireless local area network (WLAN), a cellular network, a wide area network (WAN), a metropolitan area network (MAN), or a combination of two or more such networks) over which the provider device(s) 106 and the member device(s) 110 may access the service provider computing device(s) 104 and/or communicate with one another.

The service provider computing device(s) 104 may include one or more servers or other computing devices, any or all of which may include one or more processors and memory storing computer executable instructions to implement the functionality discussed herein attributable to the medical insurance services. In various examples, the service provider computing device(s) 104 may store data associated with the member(s) 112 (e.g., member data) and the medical provider(s) 106 (e.g., medical provider data), such as in a member account or provider account, respectively. The member data may include demographic information (e.g., age, gender, ethnicity, race, occupation, marital status, etc.), member characteristics (e.g., hair color, eye color, shoe size, prosthetics, orthotics, etc.), medical history (e.g., previous diagnoses, medical procedures, surgeries, family medical history, etc.), laboratory results (e.g., glucose, cholesterol, etc.), medical test results (e.g., Echo stress test result, EKG, etc.), member location information (e.g., home address, work address, etc.), pharmacological information (e.g., prescriptions, prescription fill information (e.g., last fill, expirations, etc.), preferred pharmacy, etc.), and the like. The medical provider data may include provider location information (e.g., office locations, home address of medical provider, etc.), names and credentials of medical providers associated with a medical office and/or location, clinical visit times (e.g., average time, preferred (target) time, longest visit, shortest visit, etc.), insurance billing history, procedural history, procedural and/or practice specialties, provider quality metric (e.g., based on quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, etc.), and other information associated with the medical provider.

FIG. 1 illustrates an example in which an associate of a medical provider 108 (e.g., medical provider associate 108, medical provider 108) may submit a request for a clinical assessment 116 to the service provider computing device(s) 104 via the first instance of the application 102(1). Though illustrated as a request for a clinical assessment 116, the techniques described herein may also be applied to any other type of request submitted to the service provider computing device(s) 104, such as inquiries about insurance bills, payment information, laboratory results, and the like. In various examples, the medical provider associate 108 may launch the first instance of the application 102(1) on the provider device 106, input data associated with a clinical visit (e.g., identifying information associated with the member (e.g., name, identifier, date of birth, etc.) and/or information associated with the appointment (e.g., provider, location, date, time, etc.)), and send the request for a clinical assessment to the service provider 104.

In various examples, responsive to receiving the request for a clinical assessment 116 the service provider 104 may generate a clinical assessment 118 to be surfaced to the medical provider 108 via the first instance of the application 102(1). In some examples, the service provider computing device(s) 104 may access member data and/or medical provider data to generate the clinical assessment 118, based on the information provided in the request for a clinical assessment 116.

In various examples, the clinical assessment 118 may include one or more potential diagnoses associated with the patient, medication information, one or more gaps in care, and/or one or more clinical recommendations associated with the member care. In various examples, the clinical assessment 118 may additionally include supporting documentation associated with one or more of the potential diagnoses, medication information, gap(s) in care, and/or clinical recommendation(s). In various examples, a clinical assessment component 120 of the service provider computing device(s) 104 may be configured to receive the request for the clinical assessment and generate the clinical assessment 118.

In various examples, the medical provider associate 108 may send the request for the clinical assessment 116 to the service provider 104 responsive to receiving an indication that a member 112 is due for a clinical visit. In some examples, service provider 104 may determine that the member 112 is due for the clinical visit, such as based on member data associated with the member 112. For example, the member 112 may be eligible for annual clinical visits. Based on a determination that a time associated with the annual clinical visit is approaching, the service provider 104 may determine the member 112 is due for the clinical visit. In various examples, the service provider 104 may determine that the time is within a threshold time of the annual visit (e.g., within 30 days of a date in which the member 112 had a clinical visit the previous year). Based on the determination that the time is within the threshold time, the service provider 104 may determine the member 112 is due for the clinical visit.

In various examples, a notification component 122 of the service provider computing device(s) 104 may generate a notification 124 indicating that the member 112 is due for the clinical visit. In some examples, the service provider computing device(s) 104 may send the notification 124 to the medical provider device 106. In some examples, the notification may include a push notification, an electronic mail message, a short message system message, or any other type of message configured to surface on a device. In some examples, the notification may be provided via the first instance of the application 102(1).

In various examples, the service provider computing device(s) 104 may send the notification 124 that a member 112 is due for a clinical visit to the member computing device(s) 110. In various examples, the service provider 104 may cause the notification to surface on the member device(s) 110. Similar to the notification 124 sent to the medical provider device(s) 106, the notification 124 may include a push notification, an electronic mail message, a short message system message, or any other type of message configured to surface on a device. In some examples, the notification may be provided via the second instance of the application 102(1).

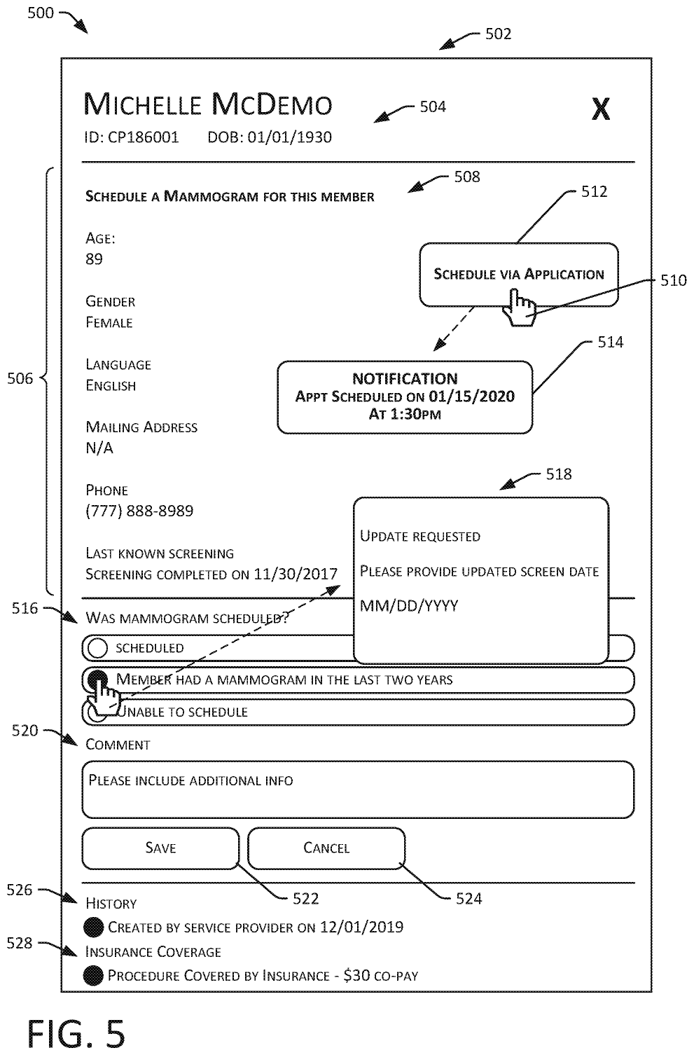

Additionally or alternatively, the service provider may determine that a member 112 is due for a screening, procedure, test, surgery consultation, or the like (collectively referred to herein as a “procedure”). In some examples, the service provider 104 may identify the procedure as a gap in care. In some examples, the gap in care may be included in a clinical assessment form, as discussed above. In other examples, the service provider 104 may cause a notification 124 to surface to the medical provider associate 108 via the first instance of the application. As will be discussed below with regard to FIG. 5, the service provider 104 may provide relevant information regarding the procedure via an interface associated with the first instance of the application 102(1). The relevant information may include member data associated with the procedure, a last known screening date, recommended care guidelines (e.g., health maintenance guidelines, recommended health screenings, etc.), and the like.

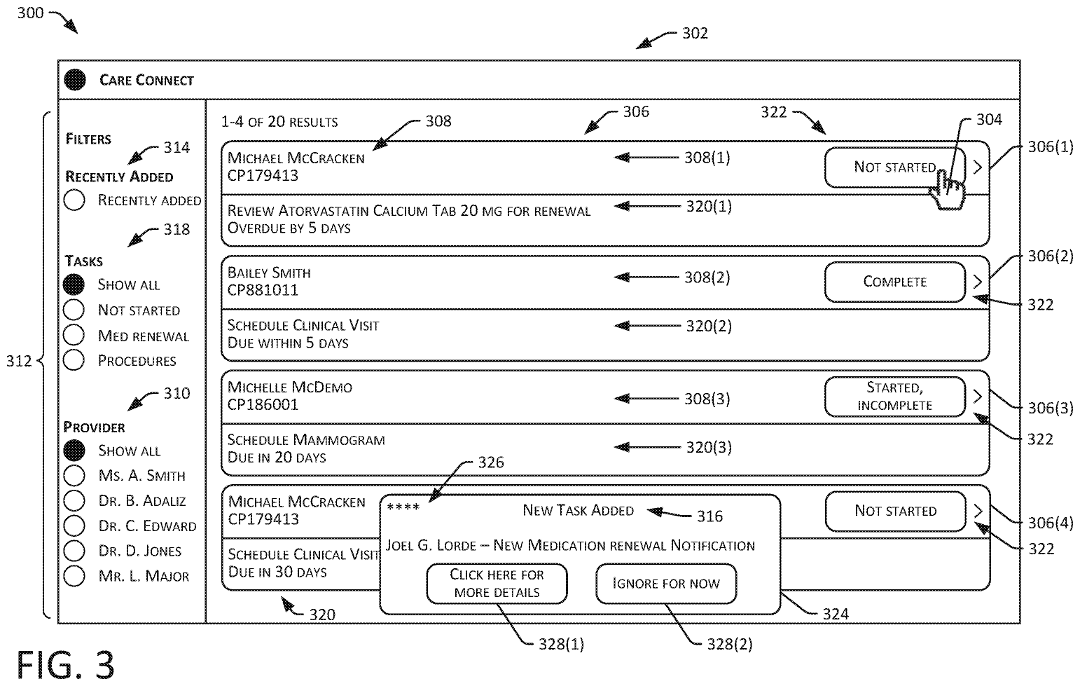

In some examples, the notification 124 associated with the procedure may surface as a push notification, electronic mail message, short message system message, or the like. In some examples, the notification 124 may surface on a care coordination task page, such as that depicted in FIG. 3. In various examples, the care coordination task page may include a means by which the medical provider associate 108 may access additional data regarding the procedure and/or initiate scheduling an appointment for the procedure with the member 112.

In various examples, responsive to at least one of the medical provider device 106 or the member computing device 110 receiving the notification 124 (regarding a clinical visit and/or a procedure), the corresponding medical provider associate 108 or member 112 may contact one another to schedule an appointment. In some examples, the appointment may be schedule via the application 102, such as via the first instance and the second instance of the application 102(1) and 102(2), respectively. In the illustrative example, the medical provider device(s) 106 and the member device(s) 110 may transmit patient data 126 (e.g., member data 126) back and forth via the application 102. In some examples, the patient data 126 may include appointment scheduling data.

In other examples, the appointment may be made over the phone or in-person. In such examples, at least one of the first instance of the application 102(1) or the second instance of the application 102(2) may be updated with the manually scheduled appointment date and time. In some examples, responsive to receiving an indication from the medical provider device that an appointment with a member 108 is scheduled, the service provider 104 may cause the second instance of the application 102(2) to be updated with the appointment information, such as via the patient data 126.

In some examples, the service provider computing device 104 may determine, based on member data, one or more medications associated with a member 112 that are expired or expiring within a threshold period of time (e.g., within a week, 14 days, 30 days, etc.). In some examples, the service provider computing device 104 may cause a notification 124 regarding the expired and/or expiring medication to surface to the medical provider associate 108 via the first instance of the application 102(1), such as via the care coordination task page. In various examples, the medical provider associate 108 may access data associated with the medication renewal and send the service provider device(s) 104 an update regarding the medication. In some examples, the update may include an indication of renewal, an indication of no renewal, a modification to a prescription (e.g., fill capacity, drug prescribed, etc.), or the like. The update may be provided to the service provider device(s) 104, such as in additional data 128.

Additionally, the service provider computing device 104 may cause the notification 124 regarding medication renewal to surface via the member device(s) 110. In some examples, responsive to receiving the notification 124, the member 112 may contact the medical provider associate 108 to renew the prescription. In some examples, the member 112 may provide information regarding the renewal to the service provider 104. In some example, the renewal information may be provided via the member device(s) 110 and/or via third-party computing device (e.g., pharmacy computing device). For example, the member 112 may receive an indication that the medical provider 108 renewed the medication, such as via a telephone conversation with the medical provider and/or associate 108, via the second instance of the application 102(2), or the like. Upon filling the renewed prescription at a pharmacy, the service provider 104 may receive an indication, from a third-party (pharmacy) computing device that the prescription has been filled. In some examples, the indication may be in the form of an insurance claim and/or request for approval for insurance payment from the third-party computing device. Based on receipt of the indication of medication renewal, the service provider computing device 104 may update the member data stored thereon, such as in a member account.

In various examples the service provider computing device 104 may be configured to process insurance claims associated with the member 112, such as via a payment processing component 130. In various examples, data associated with the insurance claims may be sent between the medical provider device(s) 106 and the service provider device(s) 104 as additional data 128. In some examples, the payment processing component 130 may receive insurance claims form the medical provider computing device(s) 106, may determine whether to approve the claims, and may cause payments to be made to the medical provider 108 based on an approval. Responsive to an approval, in some examples, the service provider computing device 104 may cause an indication of claim approval and/or processing of payment to be transmitted to at least one of the medical provider device 106 and/or the member computing device(s) 110, such as via the first instance or the second instance of the application 102(1) and 102(2), respectively.

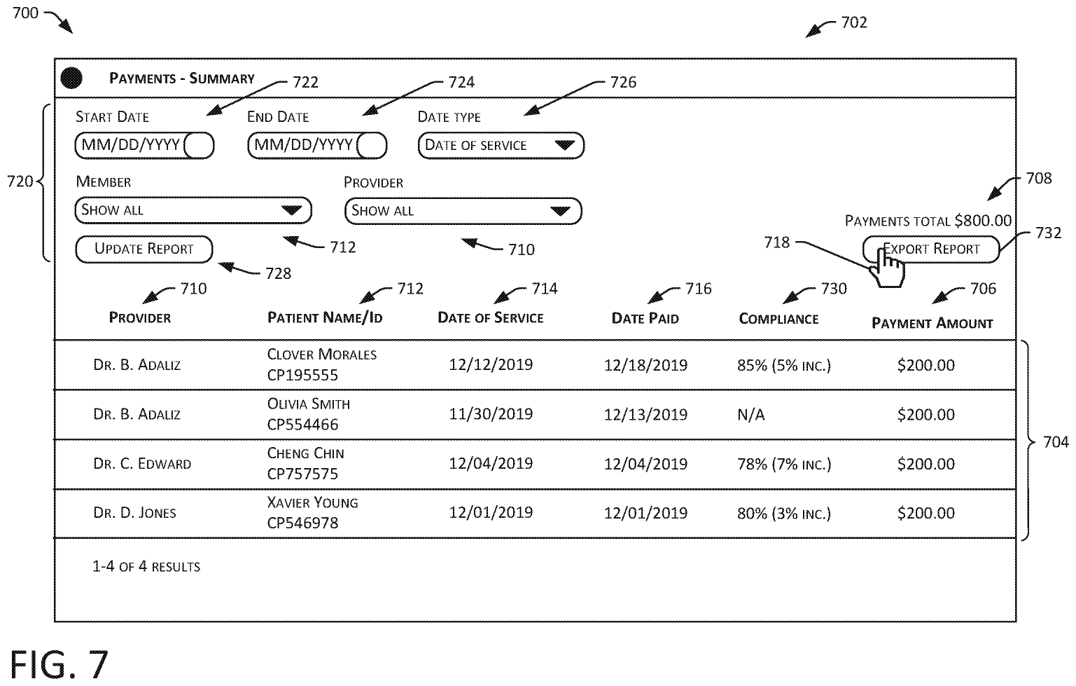

As will be discussed in greater detail below with regard to FIG. 7, the medical provider associate 108 may send a request for a financial report to the service provider computing device(s) 104. The request may include one or more members, dates, providers, and the like associated with the financial report. In some examples, the service provider computing device 104 may process the requested data and may cause the financial report to surface on the first instance of the application, such as via a payment interface. In various examples, the payment interface may provide a means by which the medical provider associate 108 may determine financial data associated with the members, the service provider, and/or the application, such as a total amount of money earned by the medical provider 108 for rendering services to members 112 associated with the service provider.

Additionally or alternatively, the additional data 128 may include member status notifications (e.g., member admitted to emergency room, released from a hospital, etc.), member scheduling notifications (e.g., appointments, procedures with other medical professionals, etc.), or the like. In various examples, the additional data 128 may be provided to the medical provider 108 (medical provider device 106) via a short-message system message, an electronic mail message, a phone call, or the like. In various examples, the additional data 128 may be surfaced on a display associated with the medical provider computing device(s) 106, such as via a push notification.

In various examples, the service provider computing device 104 may additionally be configured to provide select patient data 126 (e.g., member data 126 authorized to be transmitted to the member 112 and/or medical provider 108, based at least in part on rules and/or regulations associated with member data) directly to the member 112 via the second instance of the application 102(2). In some examples, the member data 126 may include referral information, such as based on a referral submitted by the medical provider 108. As discussed above, the member data 126 may include schedule information (e.g., scheduled clinical visits, screenings, etc.), prescription information (e.g., refills ordered, refills pending authorization, new prescriptions, etc.), insurance data, and any other information pertinent to the member 112 regarding the medical care thereof.

In some examples, the member 112 may send member data 126 to the service provider computing device(s) 104. In some examples, the member data 126 sent from the member 112 to the service provider 104 may include initial member data, such as that provided to set up an account with the service provider 104. In some examples, the member data 126 sent from the member 112 to the service provider 104 may include updated information regarding the member 112, such as care provided outside of a network associated with the service provider, schedule updates, or the like.

### Example User Interfaces

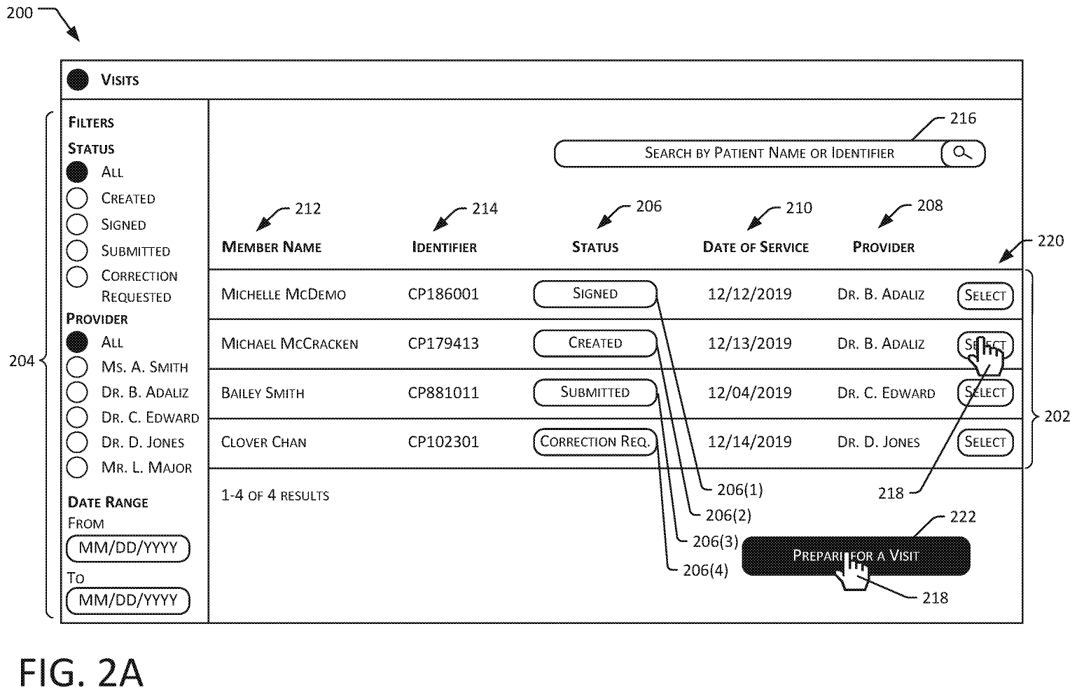

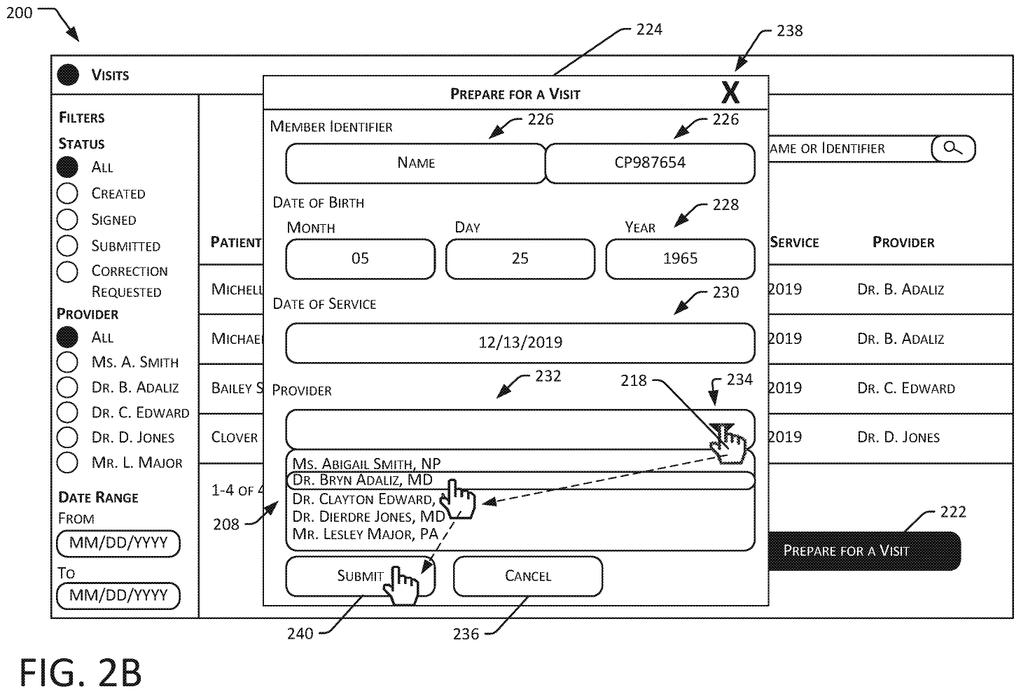

FIG. 2A—FIG. 7 are schematic views showing example user interfaces that are usable to implement the techniques described herein for providing relevant information to assist in providing effective health care management for a member. The interfaces may be generated by one or more computing device of a service provider (e.g., service provider computing device(s) 104) and transmitted to one or more medical provider computing devices (e.g., provider device(s) 106) and/or one or more member computing devices (e.g., member device(s) 110) for presentation. Additionally or alternatively, the interfaces may be generated by the provider device(s) and/or the member device(s) based at least in part on instructions received from the service provider communication device(s). As discussed above, the interfaces described in this section may, but need not, be implemented in the context of the system 100.

FIGS. 2A and 2B illustrate an example interface in which clinical assessments may be reviewed and generated. FIG. 2A illustrates an example interface 200 in which previously generated clinical assessments 202 may be reviewed. The clinical assessments 202 may include completed clinical assessments and/or data submitted by a medical provider and/or a member during a clinical visit, such as that described herein.

In various examples, the interface 200 may present the clinical assessments 202 based on one or more filters 204. In the illustrative example, the filter(s) 204 include a status of the clinical assessments 202 (e.g., status 206), a provider 208, and a date range associated with dates of service 210. In other examples, the filter(s) 204 may include other information, associated with clinical assessments, such as clinical assessments including a confirmed diagnosis, a medication modification, a new prescription, or the like.

In various examples, the status 206 of the clinical assessments 202 may include an indication of a state of the clinical assessment 202. In the illustrative example, a first status 206(1) may indicate that the associated clinical assessment 202 was signed by a provider 208, but has not been submitted. In such an example, a clinical visit may have been completed between the medical provider 208 and the member 212 and the medical provider 208 may have signed the corresponding clinical assessment 202.

As illustrated in FIG. 2A, a second status 206(2) indicates that the associated clinical assessment 202 has been created. For example, the medical provider associate 218 may have sent a request to generate the clinical assessment 202, such as that described below with regard to FIG. 2B. In response to receiving the request to generate the clinical assessment 202, the service provider may generate the clinical assessment 202 and provide an indication thereof on the interface 200 with the corresponding second status 206(2) indicating that the clinical assessment 202 was created. In such an example, the medical provider may access the clinical assessment 202 on a second instance of the application, such as on a second computing device associated with the medical provider 208, during a clinical visit between the medical provider 208 and the member 212.

In the illustrative example of FIG. 2A, a third status 206(3) indicates that the clinical assessments 202 was submitted. In such an example, a clinical visit may have been completed between the medical provider 208 and the member 212 and the medical provider 208 may have signed and submitted the corresponding clinical assessment 202.

As illustrated in FIG. 2A, the fourth status 206(4) may indicate that the clinical assessment 202 requires a correction. In such an example, the clinical assessment 202 may have been submitted, but returned due to an error (e.g., service provider determined an error or problem with the clinical assessment 202 and returned the clinical assessment for correction). In various examples, the service provider may cause the clinical assessment 202 to surface on the interface 200 with the fourth status 206(4) based on a determination that the clinical assessment 202 error is an administrative error. IN such an example, the error may include an error associated with the medical provider associate 218. In some examples, based on a determination that the error includes a clinical error or other error associated with the medical provider 208, the service provider may cause the clinical assessment to surface on another interface associated with the application, such as one associated with the medical provider 208. In such an example, the medical provider may be provided with the indication that the correction is required and may submit the corrected clinical assessment 202 to the service provider. Due to the service provider surfacing appropriate tasks to appropriate individuals associated with a medical location (e.g., medical provider 208 or medical provider associate 218), the service provider may streamline tasks, reducing a total amount of time required to complete the tasks.

In various examples, the clinical assessments 202 presented on the interface 200 may be ordered, such as based on an alphabetical order by patient name 212, based on an identifier 214, by the provider 208, or the like. In some examples, the clinical assessments 202 presented on the interface 200 may be presented in a random order.

In various examples, the interface 200 may include a search function 216, in which a medical provider associate 218 (or medical provider in the example of a single physician medical practice) may search for a particular patient name 212 or patient identifier 214. In various examples, the medical provider associate 218 may access a particular clinical assessment 202 by selecting a first selectable option 220 associated with the particular clinical assessment 202. In such examples, the medical provider associate 218 may be able to modify the clinical assessment and/or update a status 206 associated therewith.

In various examples, the medical provider associate 218 may generate a new clinical assessment 202 by selecting a second selectable option 222. Though illustrated in FIG. 2A with a corresponding label to “PREPARE FOR A VISIT,” this is merely for illustrative purposes, and any other label associated with generating a clinical assessment 202 is contemplated herein, such as “NEW,” “NEW CLINICAL ASSESSMENT,” “ADD CLINICAL ASSESSMENT,” or the like.

FIG. 2B illustrates the example interface 200 configured for generating a new clinical assessment, such as by selecting the second selectable option 222. As discussed herein, the new clinical assessment may be generated for an upcoming clinical visit between the medical provider associate 218 and a member.

In the illustrative example, responsive to selecting the selectable option 222, a window 224 may surface via the interface 200. The window 224 may include data fields 226, 228, 230, and 232 for the medical provider associate 218 to input relevant data about an upcoming clinical visit. In other examples, a new page associated with the interface 200 may launch, such as via the application or a website. In such examples, the new page may include data fields the same and/or similar to data fields 226, 228, 230, and 232.

In the illustrative example, a first data field 226 includes a member name and identifier. In various examples, the identifier may include an alphanumeric identifier, a symbol, or other indicator of a particular identifier associated with a particular member. In other examples, the first data field 226 may include either the member name or the identifier. As illustrated, the second data field 228 may include a date of birth associated with the member. In other examples, the window may include additional or alternative data fields to identify the particular member.

In the illustrative example, the third data field 230 includes a date of service associated with the clinical visit associated with the new clinical assessment, such as a date that the clinical visit is scheduled. The fourth data field 232 includes a drop-down menu option 234 for the medical provider associate 218 to select a provider 208 associated with the clinical visit. In other examples, the fourth data field 232 may include a means by which the medical provider associate 218 may type in the provider 208 associated with the clinical visit. In some examples, the fourth data field 232 may include an auto-fill option, such that the fourth data field 232 may auto-fill based on an order in which letters are typed into the data field.

In various examples, the window 224 may include a means by which the creation of the new clinical assessment may be canceled, such as by a third selectable option 236 to cancel and/or a fourth selectable option 238 (illustrated as an X). In various examples, one or both of the third selectable option or the fourth selectable option 238 may surface an option for the medical provider associate 218 to save changes. In such examples, the data input into the window 224 may be saved to complete at a later time. In some examples, the interface 200 may provide an indication of a partially complete (e.g., not yet submitted) request for a new clinical assessment.

In various examples, responsive to the medical provider selecting a fifth selectable option 240 to submit the request to generate the new clinical assessment, the service provider may generate a clinical assessment 202 and include the clinical assessment 202 on the interface 200 of FIG. 2A, such as in a created status.

FIG. 3 illustrates an example interface 300 associated with a care coordination task page 302. In various examples, the interface 300 may provide a means by which a medical provider associate 304, such as medical provider associate 108 and 218 may manage one or more care tasks 306 associated with members 308, such as members 112. Care task(s) 306 may include medication renewals, scheduling clinical visits and/or other procedures, submitting insurance claims, submitting clinical visits (e.g., sending data associated with a clinical visit to the service provider), and/or any other task related to managing the medical care of a member 308. The medical provider associate 304 may manage the task, such as by scheduling an associated appointment, receiving medical provider 310 approval to renew a prescription, and/or communicate with a service provider and/or the member 308 regarding member care.

In various examples, the service provider, such as service provider 104 may determine the care task(s) 306 based on member data. In some examples, the service provider 104 may access member data and may determine that a care task 306 associated with a member 308 is due. In some examples, the service provider may determine that a care task 306 is due based on a determination that a current date is within a threshold number of days (e.g., 12 days, 2 months, etc.) from an expiration date, a renewal date, and/or a date associated with a monthly, quarterly, semi-annual, annual, bi-annual, or the like appointment.

In some examples, the threshold number of days may be determined based on a medical provider 310 and/or medical location associated with the appointment. In some examples, the threshold number of days may be determined based on a scheduling calendar associated with the medical provider 310 and/or the medical location. In some examples, based on a determination that the medical provider 310 and/or the medical location has a full schedule (e.g., capacity above a threshold, open appointments below a threshold, etc.), the service provider may adjust the threshold number of days based on the full schedule. For example, a medical provider associate 304 may consistently schedule appointments for a medical provider 310 90 days from the date scheduled, due to a lack of earlier available appointments. Based on the 90-day scheduling requirement, the service provider may determine that a threshold number of days is 120 days, to provide the medical provider associate 304 and/or the member 308 flexibility with scheduling.

In various examples, the service provider may cause the care task(s) 306 to be presented via the interface 300. In some examples, the service provider may cause the care task(s) 306 to be presented in a random order. In some examples, the care task(s) 306 may be presented in an order determined by the service provider. In the illustrative example, the care task(s) 306 may be ordered based on time associated with the care task(s) 306 (e.g., a time in which the task 306 is due). For example, a first care task 306(1) is overdue by 5 days, a second care task 306(2) is due in 5 days, a third care task 306(3) is due in 20 days, and a fourth care task 306(4) is due in 30 days.

In some examples, the care task(s) 306 may be prioritized based on an importance associated with the task(s) 306. In some examples, the importance may be determined based on a level of severity associated therewith, such as to the health of the member 308. For example, the medication review associated with task 306(1) may be more important to the member 308(1) than the clinical visit associated with task 306(2) to the member 308(2). Accordingly, the service provider may cause the first care task 306(1) to surface above the second care task 306(2) on the care coordination task page 302. In other examples, the care task(s) 306 may be ordered alphabetically by member 308 name, numerically by member number (e.g., CP179413 associated with member 308(1), etc.), or any other way to order care task(s) 306 on the interface 300.

In various examples, the interface 300 may present the care tasks 306 based on one or more filters 312. In the illustrative example, the filter(s) 312 include recently added care tasks 314 (e.g., new task 316), a type of task 318, and a provider 310. In other examples, the filter(s) 312 may include other information, associated with medications, appointments, and the like, such as a date range, member data, or the like.

In various examples, the care task(s) 306 may provide an indication that the associated member 308 is due for a medication renewal, a clinical visit, a procedure, and/or other task associated with health care management of the member 308. In various examples, the care task(s) 306 may include an indication 320 of the associated task. For example, as illustrate in FIG. 3, the care task 306(1) may provide an indication 320(1) that the member 308(1) has an overdue medication, care task 306(2) may provide an indication 320(2) that the member 308(2) is due for a clinical visit, and care task 306(3) may provide an indication 320(3) that the member 308(3) is due for a mammogram.

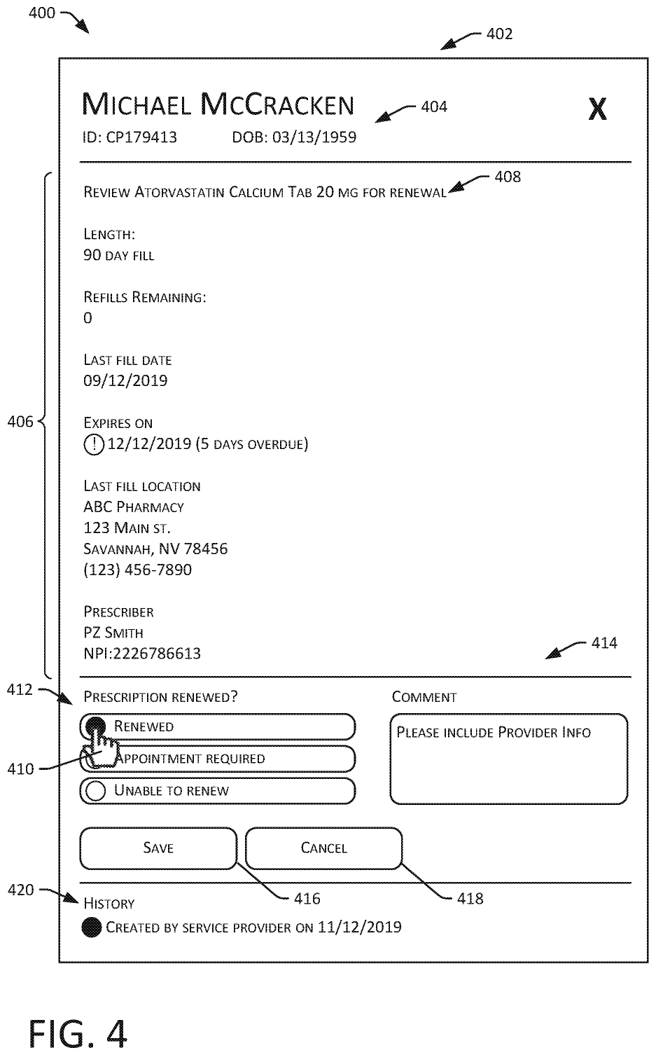

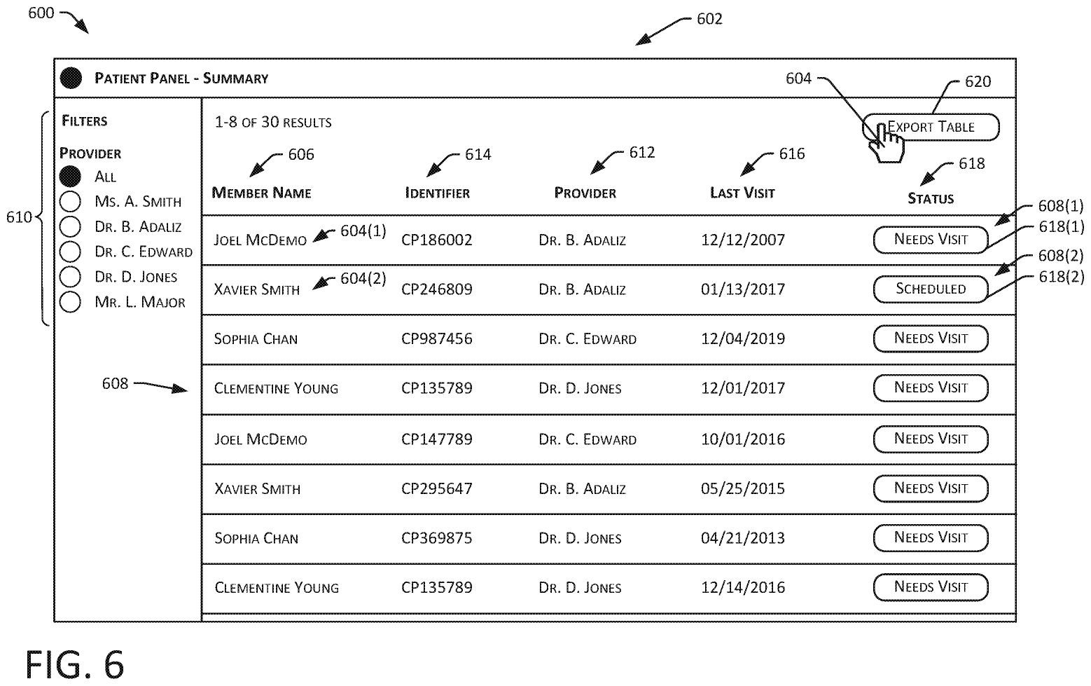

In various examples, interface 300 may provide a means by which the medical provider associate 304 may select a task 306, such as the first task 306(1), such as to renew or update information regarding the medication associated therewith. In the illustrative example, the interface 300 may include selectable option 322 that, responsive to selection, may launch a member medication review page, such as that described with regard to FIG. 4, a member procedure review page, such as that described with regard to FIG. 5, a clinical visit scheduling review page, such as that described with regard to FIG. 6, or the like.

In various examples, the selectable option 322 may provide an indication of a status of the related task 306. For example, as illustrated in FIG. 3, the first task 306(1) may include an indication that the task 306(1) has not been started, the second task 306(2) may include an indication that the task 306(2) has been completed, and the third task 306(3) may include an indication that the task 306(3) has been started but is incomplete. In various examples, a completed task, 306(2) may be presented via the interface 300 for a period of time after completion. In some examples, the period of time may include a time until a next upload of data to the service provider, a close of a business day, or the like. In such examples, the medical provider associate 304 may be able to maintain a record of tasks completed during the period of time, such as throughout a work day.

In various examples, the task(s) 306 presented via the interface 300 may be updated in real-time or near real-time. In such examples, the task(s) 306 may surface based on a determination, by the service provider, of the task(s) 306. In some examples, based on a determination of a new task 316, the service provider may include the task 316 as a task 306 presented on the interface. Additionally or alternatively, and as illustrated in FIG. 3, the service provider may cause a notification 324, such as notification 124 to surface on the care coordination task page 302 and/or another page or interface associated with the application. In the illustrative example, the notification 324 may include a member name and basic information about the notification 324, such as the new medical renewal notification, clinical visit due, or the like.

In various examples, the notification 324 may include an indication of importance 326 associated with the new task 316. In such examples, the service provider may determine the importance of the new task 316 based on a level of severity and/or time associated with the new task 316. For example, based on a determination that the new task 316 is critical to maintain the health of a member 308, the notification 324 may include four stars. In another example, based on a determination that a new task 316 is not critical to maintain the health of the member 308, the notification may include one star. For example, based on a determination that the new task 316 is time critical to maintain the health of a member 308, the notification 324 may include four stars. In another example, based on a determination that a new task 316 is not time critical to maintain the health of the member 308, the notification may include one star.

As illustrated in FIG. 3, the notification 324 may include the selectable option 328(1) to “CLICK HERE FOR MORE DETAILS” or the selectable option 328(2) to “IGNORE FOR NOW.” In some examples, responsive to selection of the selectable option 328(2), the service provider may cause the new task 316 to be added to the task(s) 306. In various examples, responsive to selection of the selectable option 328(1), the service provider may cause a member medication review page to surface, such as that depicted in FIG. 4.

FIG. 4 illustrates an example interface 400 associated with a member medication review page 402 (e.g., medication review page 402). As stated above, the member medication review page 402 may surface responsive to a selection of a selectable option 328(1) and/or selectable option 322 on the care task page 302 of FIG. 3 (e.g., selection of a selectable option related to a medication). The member medication review page 402 may include member data 404. In the illustrative example, the member data 404 includes a member name, identification number, and date of birth. In other examples, the member data 404 may include additional and/or alternative identifying information associated with the member.

As illustrated in FIG. 4, the member medication review page 402 may include medication data 406 associated with a medication (e.g., prescription) for renewal. The medication data 406 may include a medication review task 408 (e.g., medication renewal task, medication review task, etc.), such as care task 306(1). In the illustrative example, the medication review task 408 includes a care task to review medication data, such as to renew the medication. In other examples, the medication review task 408 included in the medication data 406 may include a care task to potentially modify a modification. In such examples, the modification may include an option for a medical provider 410 (e.g., medical provider associate 410, such as medical provider associate 304) to indicate a change to a prescription, such as to shift a length of the prescription (e.g., modify from 90-day fill to a 120-day fill), approve a change to a generic drug, etc.

The medication data 406 may include data associated with a prescription or other medication corresponding to the member that may be relevant to the medical provider associate 410 to complete a medication review task 408 (e.g., renew medication or indicate no renewal, review potential modification, etc.). As illustrated in FIG. 4, the medication data 406 may include a length of the prescription fill, number of refills remaining, last fill date, expiration date, last fill location, and prescriber. In other examples, the medication data 406 may include additional or alternative medication data 406, such as any other information to provide the medical provider associate 410 (medical provider) with sufficient data to inform a decision regarding renewal, appointment scheduling prior to renewal, modification, or the like.

In various examples, the medication review page 402 may include an input section 412. The input section 412 may include one or more inputs in which the medical provider associate 410 may update medication data 406, such as to indicate that a medical provider associate 410 renewed the prescription, an appointment is scheduled or required to address the medication, or that the medical provider associate 410 is unable to renew. In various examples, responsive to receiving an input that the appointment is scheduled, the service provider may update the member data, such as that stored on a member account. In various examples, responsive to receiving an input that an appointment is required, the service provider may generate a scheduling task associated with the appointment. In some examples, the service provider may cause the scheduling task to surface on the interface 400, the interface 300 or another interface associated with a medical provider computing device. In some examples, the service provider may cause the scheduling task to surface on a display of a member computing device, such as via an instance of the application of the member computing device.

In some examples, the medical provider associate 410 may be unable to review based on a lack of availability of the prescribing medical provider. In such an example, due to not being able to contact the medical provider (or a proxy), the medical provider associate 410 indicates that they are unable to renew. In various examples, the medication review page 402 may additionally include a comment section 414 in which the medical provider associate 410 may input additional information regarding the input section 412 submitted in response to whether the prescription was renewed and/or other information regarding the medication.

In various examples, the medication review page 402 may include a selectable option 416 to save the input submitted via the input section 412 and/or the comment section 414. Responsive to selection of the selectable option 416, the input may be stored on a datastore of a computing device associated with the interface 400 (e.g., medical provider computing device 106). In some examples, responsive to selection of the selectable option 416, the input may be sent to the service provider. In some examples, responsive to receipt of the input, the service provider may update the medical data associated with the member, process a renewal of the medication, generate an appointment scheduling task associated with the member (e.g., based on an indication that an appointment would first be required), and/or generate a query as to why the medication is unable to be renewed.

In various examples, the medication review page 402 may include a selectable option 418 to cancel actions related to the medication review task 408. In various examples, responsive to receiving an indication of selection of the selectable option 418, the service provider may cause any input from the input section 412 and/or comment section 414 to be discarded. In such examples, the input would not be saved in the member data.

In various examples, the medication review page 402 may include an indication of a history 420 associated with the medication review task 408. The history 420 may provide the medical provider with an indication as to when the medication review task was generated. In the illustrative example, the history 420 includes a date. In other examples, the history 420 may additionally or alternatively include other information, such as a time, a person who verified the medication review task 408, an identifier associated with the person, a contact information associated with the person, and the like. In some examples, the contact information associated with the verifier may provide the medical provider associate 410 with an individual to contact should any questions arise regarding the medication review task 408.

FIG. 5 illustrates an example interface 500 associated with a member procedure review page 502 (procedure review page 502). The procedure review page 502 may surface responsive to a selection of a selectable option 328(1) and/or selectable option 322 on the care task page 302 of FIG. 3 (e.g., selection of a selectable option related to a procedure, such as task 306(3)). The procedure review page 502 may include member data 504. In the illustrative example, the member data 504 includes a member name, identification number, and date of birth. In other examples, the member data 504 may include additional and/or alternative identifying information associated with the member.

As illustrated in FIG. 5, the procedure review page 502 may include procedure data 506 associated with a procedure review task 508 (e.g., scheduling task associated with a procedure). In the illustrative example, the procedure review task 508 includes a care task to schedule a mammogram for the member. In other examples, the procedure review task 508 may include a care task to schedule a clinical visit, a consultation, an annual physical, a cancer screening, and/or any other type of periodic appointment. In various examples, the procedure review task 508 may be determined based on a time in which a previous procedure was completed (included in the procedure data 506), a clinical recommendation or guideline with respect to the procedure, and/or member characteristics, such as those included in procedure data 506, such as member age and gender.

In various examples, service provider may determine to generate the procedure review task 508 based on a periodic interval associated with the procedure, such as a periodic interval associated with a clinical recommendation or guideline. For example, an organization associated with a particular disease may recommend a screening for a particular type of cancer once every two years. In some examples, the periodic interval may be determined based on medical provider data, such as a preference associated with the medical provider for conducting the procedure. For example, the medical provider data associated with a medical provider may include a preference to conduct a clinical visit with each member once per year. In various examples, the service provider may generate the procedure review task 508 (e.g., scheduling task associated with the procedure) based on a determination that a current time is approaching (or has exceeded) the periodic interval from a previous time associated with a last known procedure. In various examples, the procedure review task 508 may be generated based on a determination that the current time is within a threshold time of a future time associated with the periodic interval. For example, a procedure may be ordered once every two years and a threshold time for scheduling may be 14 days. The service provider may determine that the current time is within 14 days of a future date two years from completion of a last known procedure. Based on the determination that the current time is within the threshold time of a date for the procedure, the service provider may generate the procedure review task 508.

In the illustrative example, the procedure data 506 additionally include preferred language information and contact information associated with the member. In such examples, the procedure data 506 may provide a quick reference to pertinent information for a medical provider associate 510 to schedule the mammogram for the member. In various examples, the medical provider associate 510 may contact the member via the contact information, such as via the phone number included therein. In some examples, the procedure review page 502 may include a selectable option 512 to enable the medical provider associate 510 the means to schedule the procedure via an application associated with the service provider. In some examples, the application may access a calendar associated with the member, and may enable the medical provider associate 510 to select a date and/or time for scheduling the appointment. In some examples, the member may store one or more preferences with regard to scheduling appointments, such as in a member account. In such examples, the application may access the preferences, and may provide the preferred days and/or times as options to the medical provider associate 510 to schedule the appointment.

In some examples, responsive to selecting the selectable option 512, the service provider may cause a notification, such as notification 126, to surface or otherwise be presented on a member computing device, such as member computing device 110. The notification may include at least a portion of the procedure data 506 and/or one or more available dates and/or times for the procedure. Responsive to receiving an indication of selection of a date and/or time for the procedure, the service provider may update member data, and/or a calendar managed by the medical provider associate 510. In some examples, responsive to receiving an indication of selection of a date and/or time, the service provider may cause a notification 514 to surface via the procedure review page 502. In such examples, the notification 514 may provide the medical provider associate with a date and time associated with the procedure scheduled via the application. In various examples, the medical provider associate may update a calendar based in part on the notification.

In some examples, the procedure review page 502 may include an input section 516 in which the medical provider associate 510 may input data regarding whether the procedure (e.g., mammogram) was scheduled. For illustrative purposes, the input associated with the input section 516 differs from the scheduled appointment described above, such as based on a selection of the selectable option 512 and received notification 514 associated with the scheduled procedure. However, the difference is merely for illustrative purposes and is not intended to be limiting and/or conflicting. For example, based on a notification 514 that the appointment was scheduled, the medical provider associate 510 may input an indication that the appointment was scheduled in the input section 516.

In some examples, the input section 516 may include an option to indicate that the member had a procedure within a time period. In the illustrative example, the time period includes the past two years (e.g., 24 months). In some examples, the indicated period may be determined by the service provider, such as based on clinical guidelines, medical provider preferences (e.g., medical provider indicates a preference to conduct an annual physical exam on patients older than 65), insurance policy terms (e.g., semi-annual check-ups, etc.), or the like.

As illustrated in FIG. 5, responsive to providing input via the input section 516, the service provider may cause a window 518 to surface or otherwise be presented on the procedure review page 502. The window 518 may request additional information regarding the input submitted via the input section 516. For example, responsive to indicating that the member conducted the procedure within the time period (e.g., mammogram completed within the last two years), the service provider may cause the window 518 to surface requesting a date associated with the screening. Responsive to receiving the input via the window 518, the service provider may update member data. Though illustrated as a date (e.g., month, date, and year) associated with the screening, that is not intended to be so limiting, and other information may additionally or alternatively be requested, such as month and year, medical provider information, medical location information associated with the procedure, and the like. For another example, responsive to indicating that the procedure was scheduled, the window 518 may include a request for appointment information, such as date, time, provider, and the like. For yet another example, responsive to an input that the medical provider associate 510 is unable to schedule the appointment, the window 518 may include a request for a justification for not scheduling the appointment (e.g., member unavailable, member out of town, etc.).

Additionally or alternatively, the procedure review page 502 may include a comment section 520 in which the medical provider associate 510 may input additional information regarding the procedure, an appointment associated therewith, the member, or the like. For example, the medical provider associate 510 may indicate in the comment section 520 that the member has indicated a preference for a female provider for the procedure. Responsive to receiving the input via the comment section 520, the service provider may update the member data to reflect the member preference.

In various examples, the procedure review page 502 may include a selectable option 522 to save the input submitted via the input section 516, the window 518, and/or the comment section 520. Responsive to selection of the selectable option 522, the input may be stored on a datastore of a computing device associated with the interface 500 (e.g., medical provider computing device 106). In some examples, responsive to selection of the selectable option 522, the input may be sent to the service provider. In some examples, responsive to receipt of the input, the service provider may update the medical data associated with the member, update a calendar associated with the member, send a notification of the scheduled appointment to the member, update an appointment scheduling task associated with the member (e.g., based on an indication that the procedure could not be scheduled), and/or generate a query as to why the appointment could not be scheduled.

In various examples, the procedure review page 502 may include a selectable option 524 to cancel actions related to the procedure review task 508. In various examples, responsive to receiving an indication of selection of the selectable option 524, the service provider may cause any input from the input section 516, the window 518, and/or comment section 520 to be discarded. In such examples, the input would not be saved in the member data.

In various examples, the procedure review page 502 may include an indication of a history 526 associated with the procedure review task 508. The history 526 may provide the medical provider with an indication as to when the procedure review task 508 was generated. In the illustrative example, the history 526 includes a date. In other examples, the history 526 may additionally or alternatively include other information, such as a guideline associated with the procedure review task 508, a time associated with the procedure review task 508, a person who verified the procedure review task 508, an identifier associated with the person, a contact information associated with the person, and the like. In some examples, the contact information associated with the verifier may provide the medical provider associate 510 with an individual to contact should any questions arise regarding the procedure review task 508.

In various examples, the procedure review page 502 may include insurance coverage information 528. In the illustrative example, the insurance coverage information 528 indicates that the procedure is covered by a medical insurance program associated with the member and a co-pay associated with the procedure. In other examples, the insurance coverage information 528 may indicate that the procedure is fully covered (e.g., no co pay) or not covered by the medical insurance program. In some examples, the insurance coverage information 528 may include additional or alternative information, such as a date associated with insurance coverage verification, a total amount covered by the insurance company, a pre-approved location associated with the procedure, such as for a referral, and/or any relevant insurance information that may inform a decision for the member to conduct the procedure and/or for the medical provider associate 510 to schedule the procedure.

FIG. 6 illustrates an example interface 600 associated with a clinical visit scheduling review page 602 (e.g., clinical visit page 602). In various examples, the clinical visit page 602 may provide a quick reference for a medical provider associate 604 to determine one or more member(s) 606 associated with clinical visit tasks 608 (e.g., need clinical visit, scheduled clinical visits, etc.). Although illustrated as a clinical visit scheduling review page 602, this is merely for illustrative purposes and is not intended to be so limiting. For example, other types of scheduling review pages are contemplated herein, such as a review page associated with procedures to be scheduled and/or scheduled (e.g., procedures due for members), a combination of clinical visits and procedures, and the like. Accordingly, as described herein the clinical visit tasks 608 may represent clinical visits to be scheduled and/or procedures (e.g., screening, procedure, test, surgery, consultation, etc.) to be scheduled for a member.

In the illustrative example, the clinical visit page 602 includes eight (8) clinical visit tasks 608 associated with different members 606. In other examples, the clinical visit page 602 may include a greater or lesser number of clinical visit tasks 608. In some examples, the clinical visit page 602 may include two or more clinical visit tasks 608 associated with a single member 606. For example, the member 606 may require monthly visits. Accordingly, the clinical visit page 602 may include a clinical visit tasks 608 for a first month and a second clinical visit task 608 for a second month, etc.

In various examples, clinical visit tasks 608 may be associated with a clinical visit schedule (e.g., periodic scheduling of clinical visits). In such examples, the service provider may determine one or more tasks 608 based on the clinical visit schedule. In some examples, the clinical visit schedule may be determined based on a one or more factors. The factor(s) may include member data associated with members 606 (e.g., age, diagnosis, member preference, etc.), a medical provider preference, an insurance plan associated with a member 606, and the like. For example, members 606 above a threshold age may be scheduled for annual clinical visits. For another example, members 606 with a particular diagnosis may be scheduled for a clinical visit every quarter.

In various examples, the interface 600 may present the clinical visit tasks 608 based on one or more filters 610. In the illustrative example, the filter(s) 610 include a provider 612 associated with the clinical visit tasks 608 (e.g., associated with the clinical visit). In other examples, the filter(s) 610 may include other information associated with clinical visits, such as a date range, scheduled visits, visits needed, and the like.

In various examples, the clinical visit tasks 608 may be presented via the clinical visit page 602 in a random order. In some examples, the clinical visit tasks 608 may be presented in a determined order. The determined order may include an alphabetical order of the members 606, numerical order associated with member identifiers 614, a date order associated with a last visit 616, a status indicator 618 associated with the clinical tasks 608, and/or other clinical task 608 ordering scheme.

In various examples, the clinical visit tasks 608 may include a status indicator 618, such as the first status indicator 618(1) and a second status indicator 618(2) that provide a status 618 of the respective clinical visit tasks 608(1) and 608(2). As illustrated in FIG. 6, the status indicator 618(1) may provide an indication that a clinical visit needs to be scheduled (e.g., clinical visit task 608(1) is to schedule a clinical visit) and the status indicator 618(2) provides an indication that a clinical visit is scheduled (e.g., clinical visit task 608(2) scheduling complete). In other examples, the status indicator 618 may provide an indication that the clinical visit is overdue, a time frame associated with scheduling (e.g., needs a visit within the next 30 days, etc.), and the like.

In various examples, the status indicator 618 may include a link to schedule a visit, such as based on a first status indicator 618(1) including an indication that the member needs a clinical visit. In such examples, the link may provide a medical provider associated with a means to schedule the appointment through the application associated with the interface and/or may provide the medical provider associate 604, such as described above with regard to FIG. 3. In some examples, the status indicator 618 may include a link to view data associated with a needed visit (e.g., clinical visit task 608(1) with associated indicator 618(1). In such examples, responsive to selection of the first status indicator 618(1), the service provider may surface or present a procedure review page, such as procedure review page 502 of FIG. 5, associated with a clinical visit. In some examples, the second status indicator 618(2) associated with a scheduled clinical visit may include a link to view data associated with the scheduled clinical visit, such as a date, time, etc. associated with the scheduled visit.

In various examples, the clinical visit page 602 may include a selectable option 620 to provide a means by which the medical provider associate 604 may export the table, such as to save the data associated with clinical visit tasks 608.

FIG. 7 illustrates example interface 700 associated with a payment review page 702 including one or more payment summaries 704. In various examples, the payment summary 704 may include data associated with services rendered, such as medical services. In various examples, the payment review page 702 may include a financial report associated with services rendered and/or payments received from at least one of a service provider or a member. In some examples, the financial report may include a payment amount 706 associated with individual payment summaries 704 and/or a total amount 708 associated with a group of payment summaries 704, such as those associated with a particular financial report.

In the illustrative example, the payment summary 704 includes a medical provider 710 associated with the service, one or more members 712 (e.g., name and/or identifier), a date (or range of dates) the service was rendered 714, a date the payment was rendered 716, and the payment amount 706. In other examples, the payment summary 704 may include additional or alternative information, such as co-pay information, date of co-pay receipt, and the like.

In various examples, a medical provider associate 718 may generate the financial report utilizing one or more filters 720. In the illustrative example, the filter(s) 720 include a medical provider 710 associated with the service (e.g., associated clinical visit, procedure, etc.), a start date 722 and an end date 724 representing a date range for the financial report, the date range corresponding to dates in which services were rendered, a date type 726 (e.g., date of service, date paid, etc.), members 712 associated with the services, provider(s) 710 associated with the services, and the like. In other examples, the filter(s) 720 may include other information associated with the payments 704, such as payments including co-pays, and the like.

In various examples, the medical provider associate 718 may submit a request for a new financial report and/or update the presented financial report associated with the payment review page 702 by selecting the selectable option 728. In the illustrative example, the selectable option 728 includes a label “UPDATE REPORT.” In other examples, the selectable option 728 may include a different label associated with applying the filters 720. In various examples, responsive to receiving an indication that the medical provider associate 718 selected the selectable option 728 and one or more filter 720 settings (e.g., start date 722, end date 724, a date type 726, a member 712, and/or a provider 710), the service provider may generate the financial report displayed on the payment review page 702 and may cause the financial report to surface or otherwise be presented via the interface 700.

Additionally or in the alternative, the service provider may be configured to provide the medical provider with an indication of compliance 730 associated with medications and/or procedures, such as based on the scheduling tasks and medication renewal tasks described herein. In some examples, the indication of compliance 730 may include a compliance rate associated with clinical visits, procedures, and/or medication renewals, fills, etc. In some examples, the indication of compliance may include an increase or decrease in compliance of the associated procedure and/or medication renewal over a period of time. The period of time may include a month, a year, a time since associating with the service provider, or other time period. In various examples, the indication of compliance 730 may provide the medical provider with an indication of an improvement in health care services provided to members resulting from the services provided by the service provider.

In some examples, and for some procedures and/or medication renewal tasks, a compliance therewith may not be applicable. Accordingly, a particular payment summary 704 may indicate that a compliance 730 associated therewith is not applicable.

In various examples, the medical provider associate 718 may export the report, such as by selecting the export report selectable option 732. In such examples, the medical provider associate 718 may export to save, print, or perform another function with regard to the financial report (and/or compliance data).

### Example Computing Architecture

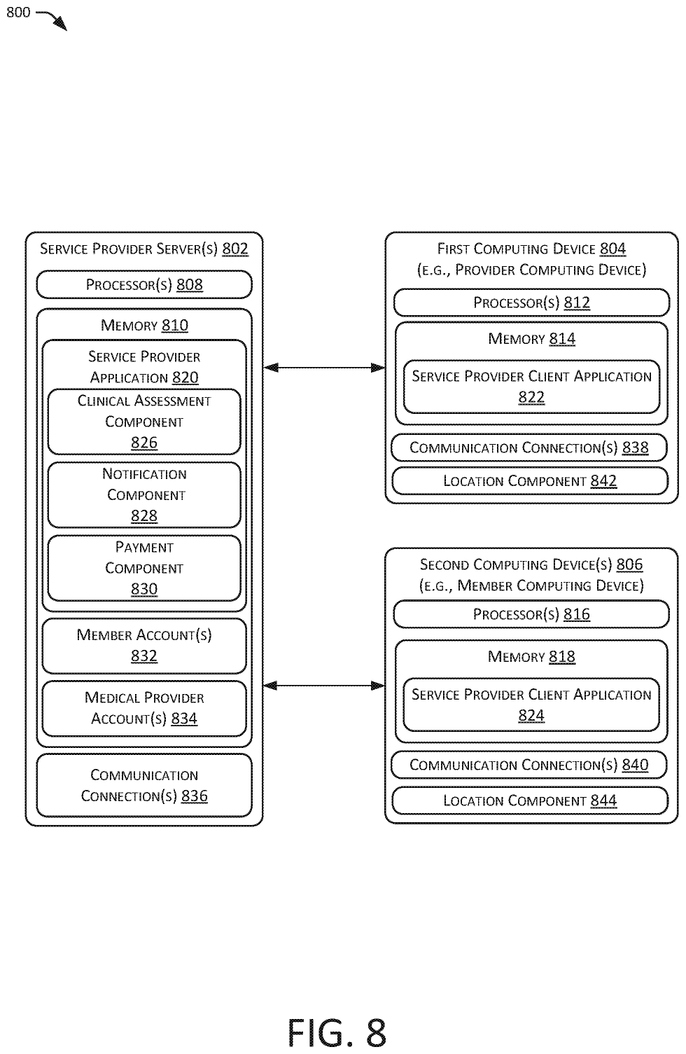

FIG. 8 illustrates a block diagram illustrating an example system 800 of computing devices usable to implement example techniques described herein. For example, FIG. 8 illustrates example computing devices including service provider server(s) 802, one or more first computing devices 804, and one or more second computing devices 806, that interact over a network, such as network 114 in FIG. 1. By way of example and not limitation, the service provider server(s) 802 may be representative of servers used to implement the system 100, the first computing device(s) 804 may be representative of the medical provider computing device 106 associated with the medical provider 108, and the second computing device(s) 806 may be representative of the member computing device 110 associated with the member 112.

The service provider server(s) 802 may comprise one or more individual servers or other computing devices that may be physically located in a single central location or may be distributed at multiple different locations. The service provider server(s) 802 may be hosted privately by an entity administering all or part of the communications network (e.g., a utility company, a governmental body, distributor, a retailer, manufacturer, etc.), or may be hosted in a cloud environment, or a combination of privately hosted and cloud hosted services.

Each of the computing devices described herein may include one or more processors and/or memory. Specifically, in the illustrated example, service provider server(s) 802 include one or more processors 808 and memory 810, first computing device(s) 804 includes one or more processors 812 and memory 814, and second computing device(s) 806 includes one or more processors 816 and memory 818. By way of example and not limitation, the processor(s) may comprise one or more Central Processing Units (CPUs), Graphics Processing Units (GPUs), or any other device or portion of a device that processes electronic data to transform that electronic data into other electronic data that may be stored in registers and/or memory. In some examples, integrated circuits (e.g., ASICs, etc.), gate arrays (e.g., FPGAs, etc.), and other hardware devices may also be considered processors in so far as they are configured to implement encoded instructions.

The memory may comprise one or more non-transitory computer-readable media and may store an operating system and one or more software applications, instructions, programs, and/or data to implement the methods described herein and the functions attributed to the various systems. In various implementations, the memory may be implemented using any suitable memory technology, such as static random-access memory (SRAM), synchronous dynamic RAM (SDRAM), nonvolatile/Flash-type memory, or any other type of memory capable of storing information. The architectures, systems, and individual elements described herein may include many other logical, programmatic, and physical components, of which those shown in the accompanying figures are merely examples that are related to the discussion herein.

As shown in FIG. 8, service provider server(s) 802 include a service provider application 820, first computing device(s) 804 includes service provider client application 822, and second computing device(s) 806 includes service provider client application 824 that enables interaction of content among the computing devices via the service provider server(s) 802. For example, content (e.g., member data, medical provider data, scheduling data, referral data, insurance data, payment data, etc.) can be shared among users associated with service provider accounts (e.g., member accounts, medical provider accounts, etc.) of an insurance provider network provided by the service provider system and may include sharing content in accordance with an account of a user that is restricted, such as based on health information privacy rules and/or regulations. In some examples, the service provider client application enables interfaces to access content, to view content, and to generate content as those described with reference to FIGS. 2A-7, for example. In particular examples, service provider server(s) 802 may send instructions to present, transmit, and receive content as discussed with reference to FIG. 2A-FIG. 7.

FIG. 8 further illustrates service provider server(s) 802 as including clinical assessment component 826, notification component 828, and payment component 830 to enable content such as clinical assessments, scheduling notifications, medication renewal notifications, financial reports, and the like, to be shared among the computing devices. In various examples, the clinical assessment component 826 may be configured to generate on or more clinical assessments associated with a member. In various examples, the clinical assessment component 826 may receive a request to generate a clinical assessment, such as from a medical provider associate (e.g., office staff, scheduler, medical provider, etc.). The medical provider associate may provide identifying information about a member, such as a member name, date of birth, identifier, or other member data in the request for the clinical assessment. Responsive to receipt of the request, the clinical assessment component 826 may determine one or more potential diagnoses, medications, gaps in care, and/or clinical recommendations associated with the member. The clinical assessment component 826 may generate the clinical assessment including the potential diagnoses, medications, gaps in care, and/or clinical recommendations and may provide the clinical assessment to the medical provider via the service provider client application 822 of the first computing device 804.

In various examples, the notification component 828 may be configured to determine one or more medications for a member that are due to be renewed. In various examples, the notification component 828 may access member data associated with a member, such as that stored on a member account 832. The member data may include one or more current medications (prescriptions, over-the-counter medications consumed by the member, etc.), a medication history (e.g., expired prescriptions, previously consumed over-the-counter medications, etc.), clinical guidelines for medications based on a diagnosis of the member, one or more medication modifications (e.g., generic drug availability for a current medication, fill length modification, etc.), and the like.

The notification component 828 may determine, based on the member data, that a medication is due for renewal. In some examples, the determination may be based on an expiration date being within a threshold time of a current date (e.g., within 5 days, 7 days, etc.). In some examples, the threshold time may be determined based on the member, the medical provider, and/or the level of severity associated with the medication (e.g., importance of the medication to member health). In various examples, the notification component 828 may cause a notification of the medication renewal to surface or otherwise be presented on the first computing device 804, such as via the service provider client application. In some examples, the notification component 828 may cause the notification to surface on the second computing device 806, such as via the service provider client application 824, to inform the member of the medication expiration. In such examples, the member may be able to proactively contact the medical provider to receive an authorization for medication renewal. In various examples, the notification of the medication renewal may surface as care task for the medical provider associate to complete, such as care task 306 of FIG. 3.

In some examples, the notification component 828 may determine, based on the member data, that a modification to a medication is available. In such examples, the notification component 828 may generate a notification to surface via the service provider client application 822, informing the medical provider of the medication modification availability. In various examples, the notification may surface as a care task, such as care tasks 306 of FIG. 3. In such examples, the medication modification may surface to a medical provider associate to address, such as to schedule an appointment, request the medical provider to review, or the like.

In various examples, the notification component 828 may be configured to determine one or more procedures for a member that are due to be renewed. As used herein, the procedures may include clinical visits, screenings, medical procedures, consultations, and the like. In various examples, the notification component 828 may determine the procedures based on a medical history associated with a member, such as that stored on a member account 832. The notification component 828 may generate a notification to inform the medical provider of the procedure for the member. In various examples, the notification of the procedure may surface as care task for the medical provider associate to complete, such as care task 306 of FIG. 3. In some examples, the procedure may surface as a gap in care, such as in a clinical assessment form generated by the clinical assessment component 826 and provided to the service provider client application for use during a clinical visit between a medical provider and the member.

In various examples, the payment component 830 may receive an indication that a member is due for a medication renewal and/or due for a procedure, such as from the notification component 828. Based on the indication, the payment component 830 may process a pre-approval for the medication renewal and/or procedure. A pre-approval may include a guarantee to that the service provider will pay for the medication and/or procedure. In various examples, the pre-approval may streamline the member care, reducing delays associated with processing insurance approvals. In various examples, the payment component 830 may send an indication of pre-approval to the first computing device 804 and/or the second computing device 806, such as via the service provider client applications 822 and 824, respectively.

In some examples, the payment component 830 may be configured to process insurance claims and provide payments to the medical provider, such as based on services rendered to a member. In some examples, the payment component 830 may determine an approval associated with a service rendered for a member and may cause a payment associated therewith to be provided to the service provider client application 822. In some examples, based on a determination of approval (or disapproval), the payment component 830 may send an indication thereof to the first computing device 804 and/or the second computing device 806, such as via the service provider client applications 822 and 824, respectively. In some examples, the payment component 830 may cause the indications to be stored in a member account 832 associated with the member and/or a medical provider account 834 associated with the medical provider.

In various examples, the payment component 830 may generate financial reports associated with payments pending and/or rendered. In various examples, the financial reports may be generated responsive to a request from the first computing device 804, such as via the service provider client application 822. In some examples, the request may include one or more filters to specify a date range, date type, provider, member, and/or other data associated with the financial report, such as that described above with regard to FIG. 7. The payment component 830 may provide the financial report to the service provider client application 822 for review by the medical provider (and/or associate thereof).

As shown in FIG. 8, service provider server(s) 802 may include communications connection(s) 836, first computing device(s) 804 may include communications connection(s) 838, and second computing device(s) 806 may include communications connection(s) 840 that enable communication between at least the service provider server(s) 802 and one or more of the first computing device(s) 804, and the second computing device(s) 806.

The communication connection(s) 836, 838, and/or 840 may include physical and/or logical interfaces for connecting service provider server(s) 802, first computing device(s) 804, and/or second computing device(s) 806 to another computing device or a network, such as network(s) 114. For example, the communications connection(s) 836, 838, and/or 840 may enable Wi-Fi-based communication such as via frequencies defined by the IEEE 802.11 standards, short range wireless frequencies such as Bluetooth®, cellular communication (e.g., 2G, 2G, 4G, 4G LTE, 5G, etc.) or any suitable wired or wireless communications protocol that enables the respective computing device to interface with the other computing device(s).

As shown in FIG. 8, the first computing device(s) 804 may include a location component(s) 842, and second computing device(s) 806 may include location component(s) 844 that enable the respective computing device(s) 804 and 806 to determine a location associated therewith. The location component(s) 842 and/or 844 may include one or more of a GPS component, cellular identification component, inertial sensor, Bluetooth beacon, or other component for determining a location of the respective computing device 804 or 806. In some examples, the service provider server(s) 802 may send a request for a current location to the first computing device 804 or the second computing device 806. The location component 842 or 844 may determine the current location and cause the respective computing device 804 or 806 to send the current location to the service provider server(s) 802.

While FIG. 8 is provided as an example system 800 that can be used to implement techniques described herein, the techniques described and claimed are not limited to being performed by the system 800, nor is the system 800 limited to performing the techniques described herein.

### Example Methods

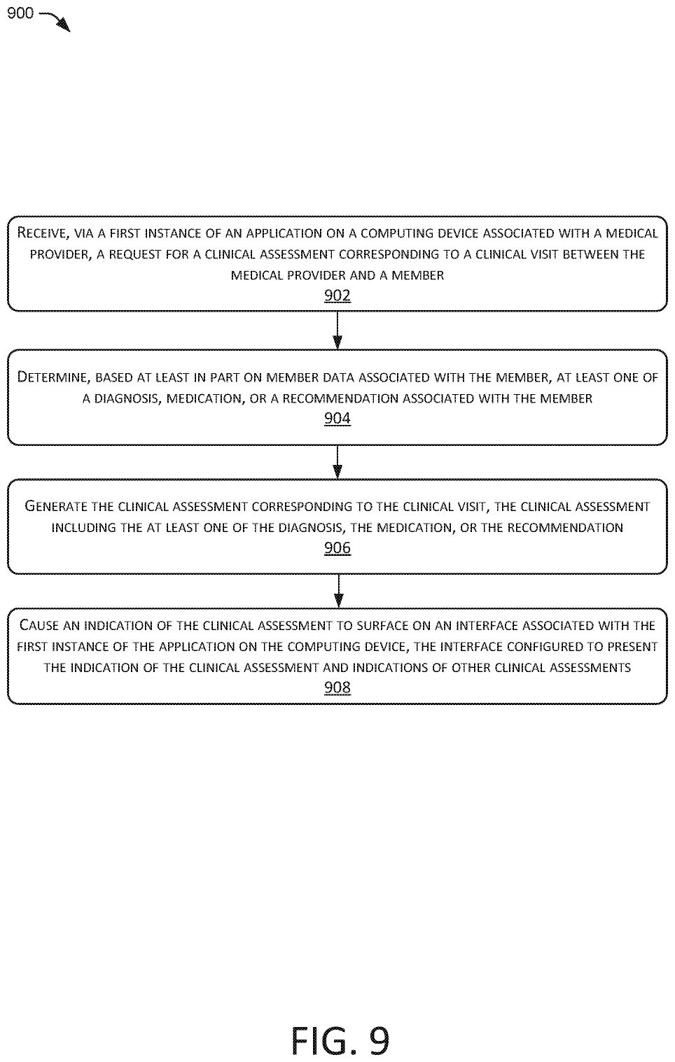

FIGS. 9-12 illustrate example processes in accordance with embodiments of the disclosure. These processes are illustrated as logical flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes.

FIG. 9 illustrates an example processes 900 for providing a clinical assessment to a medical provider, utilizing the techniques described herein. In some instances, some or all of process 900 may be performed by one or more components in the systems 100 or 800. By way of example and not limitation, the service provider computing device (e.g., service provider) referred to in process 900 may be representative of a computing device associated with the service provider 104 or service provider server(s) 802, the medical provider computing device (e.g., member device) referred to in process 900 may be representative of the medical provider computing device(s) 106 and/or first computing device(s) 804 and the member computing device referred to in process 900 may be representative of the member computing device(s) 110 and/or the second computing device(s) 806. However, the process 900 is not limited to being performed by the system 100 or 800.

At operation 902, the process 900 may include receiving, via a first instance of an application on a first computing device associated with a medical provider, a request for a clinical assessment corresponding to a clinical visit between the medical provider and a member, such as that described above with regard to FIG. 2B. In some examples, the request may include member data, such as that required to identify the member (e.g., name, date of birth, identifier, etc.), a date of service (e.g., date associated with the clinical visit), and the like.

At operation 904, the process 900 may include determining, based at least in part on member data associated with the member, at least one of a diagnosis, medication, or a recommendation associated with the member. The diagnosis may include a potential diagnosis associated with the member, as determined based on member data. In some examples, the diagnosis may be determined utilizing machine learning techniques. In some examples, the medication may include one or more current medications associated with the member. The medication may include a medication that due to be renewed, a medication that is eligible for a modification (e.g., generic drug available, prescription length modification, etc.), and/or a medication to be reviewed by the medical provider during a clinical visit. The recommendation may include a recommendation to modify the medication, a recommendation for the medical provider to order a procedure for the member, such as based on a gap in care (e.g., missing procedure in member care history, such as based on clinical guidelines), and/or a clinical recommendation for the medical provider to discuss with the member. The clinical recommendation may include a procedure, potential new medication, medication modification based on clinical guidelines, statistics associated with the member (e.g., based on demographics, a current diagnosis, etc.) and/or other recommendation provided to the medical provider with respect to a particular member.

At operation 906, the process 900 may include generating the clinical assessment corresponding to the clinical visit, the clinical assessment including the at least one of the diagnosis, the medication, or the recommendation. In various examples, the clinical assessment may provide the medical provider with relevant information associated with the member, such as to assist the medical provider in maximizing effectiveness and efficiency associated with the clinical visit (e.g., maximize care, minimize time).

At operation 908, the process 900 may include causing an indication of the clinical assessment to surface or otherwise be presented on an interface associated with the first instance of the application on the computing device, the interface configured to present the indication of the clinical assessment and indications of other clinical assessments. In various examples, the interface may include a care task page, such as interface 600 associated with the clinical visit scheduling review page 602 of FIG. 6. Though this is merely an illustrative example, and other interfaces may include the indication of the clinical assessment, such an interface including scheduled appointments associated with a medical provider for a particular date or range of dates, and the like.

In various examples, the indication of the clinical assessment may include a selectable option to view the clinical assessment. In such examples, the medical provider and/or an associate thereof may access the clinical assessment by selecting the selectable option. The medical provider and/or associate thereof may review the contents of the clinical assessment, such as to determine a length of a visit, recommended procedures (e.g., gaps in care), and the like.

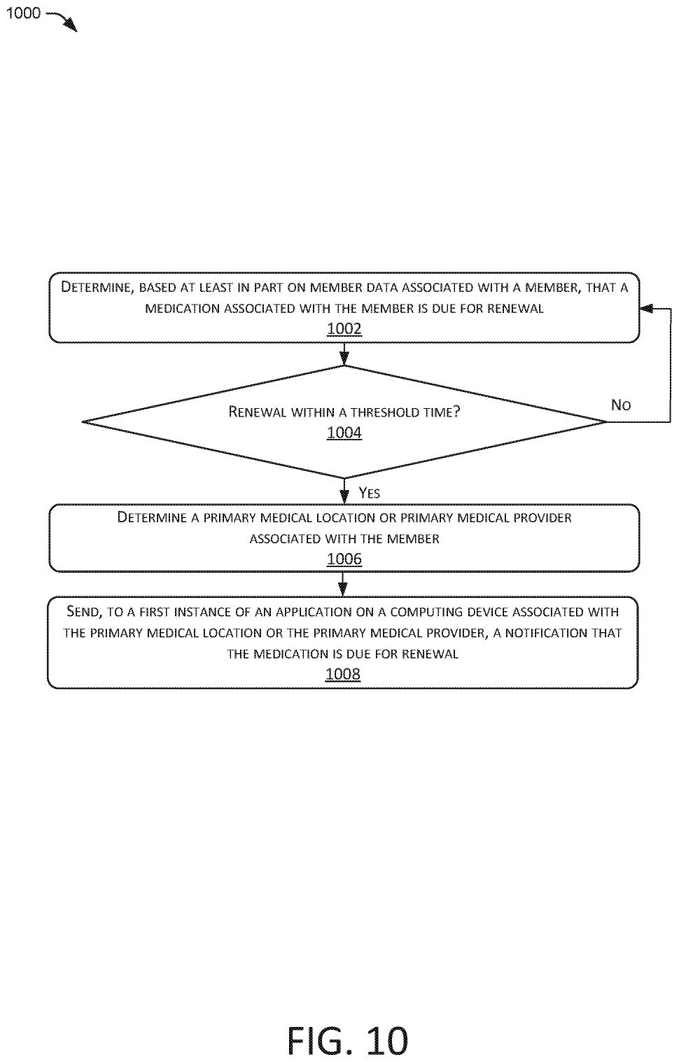

FIG. 10 illustrates an example process 1000 for determining that a member is due for a medication renewal and sending a notification of the medication renewal to a medical provider associated with the member, utilizing the techniques described herein. In some instances, some or all of process 1000 may be performed by one or more components in the systems 100 or 800. By way of example and not limitation, the service provider computing device (e.g., service provider) referred to in process 1000 may be representative of a computing device associated with the service provider 104 or service provider server(s) 802, the medical provider computing device (e.g., member device) referred to in process 1000 may be representative of the medical provider computing device(s) 106 and/or first computing device(s) 804 and the member computing device referred to in process 1000 may be representative of the member computing device(s) 110 and/or the second computing device(s) 806. However, the process 1000 is not limited to being performed by the system 100 or 800.

At operation 1002, the process 1000 may include determining, based at least in part on member data associated with a member, that a medication associated with the member is due for renewal. In various examples, a service computing device may determine that the medication is due for renewal based on member data associated with the member. The member data may include medication data, such as current medications (e.g., fill length, prescription date, fill date, expiration date, etc.), a medication history, fill history, fill location, and the like.

At operation 1004, the process 1000 may include determining whether the renewal is within a threshold time. The threshold time may include any length of time determined by the service provider, such as 24 hours, 4 days, 12 days, or the like. In some examples, the threshold time may be based in part on the medication length. In such examples, the service provider may access the medication length in the member data and determine the threshold time based on the medication length. In various examples, the threshold time may be determined based on an average time for the member and/or an average time associated with multiple members to refill a medication. In such examples, the service provider may access member data associated with the member and/or a plurality of members to determine the average time. In some examples, the threshold time may be determined based on a medical provider preference.

In some examples, the threshold time may be determined based on a level of importance (e.g., level of severity) associated with the medication. In such examples, medications with associated high levels of importance (e.g., critical to member health) may include a higher threshold time as compared to medications with associated lower levels of importance (e.g., elective prescriptions). For example, a service provider may assign a low level of importance (e.g., 3 on a scale of 10) to an acne medication and a high level of importance (e.g., 8 on the scale of 10) to a blood pressure medication. Based on a determination that the acne medication has a low level of importance, the service provider may determine a threshold time of 3 days, and based on a determination that the blood pressure medication has a high level of importance, the service provider may determine the threshold time of 8 days, to provide the medical provider additional time to renew the medication and the member additional time to refill the medication.

Based on a determination that the renewal is not within the threshold time (“No” at operation 1004), the process 1000 may include, determining that the medication associated with the member is due for renewal, such as that described above with respect to operation 1002. In some examples, the service provider may continuously and/or periodically (e.g., daily, weekly, etc.) monitor the expiration associated with the medication to determine when to provide a notification of the approaching renewal.

In some examples, the service provider may receive an indication that the medication has been renewed, such as via a clinical assessment or via a medication review page, such as medication review page 402 associated with interface 400 of FIG. 4. Based on the indication, the service provider may update the member data.

Based on a determination that renewal is within the threshold time (“Yes” at operation 1004), the process 1000 may include, at operation 1006, determining a primary medical location or primary medical provider associated with the member. In some examples, the primary medical location or primary medical provider may be determined based on a determination of a primary care physician (or other provider) associated with the member. In such examples, the primary care physician may be stored in the member data associated with the member.

In some examples, the primary medical location or primary medical provider may be determined based on a type of medication that is due for renewal. In some examples, based on a determination that the medication is a specialty medication, the primary location and/or primary medical provider may include a medical provider associated with the specialty. For example, based on a determination that the medication is for a heart arrythmia, the service provider may determine a primary medical location or primary medical provider associated with a heart specialty clinic and/or heart surgeon.

At operation 1008, the process 1000 may include sending, to a first instance of an application on a computing device associated with the primary medical location or the primary medical provider, a notification that the medication is due for renewal. In some examples, the notification may surface or otherwise be presented as a care task, such as care task 306 of FIG. 3. In some examples, the notification may surface or otherwise be presented as an electronic mail message, a short message system message, a message via the application, and/or a pop-up notification via the first instance of the application, such as notification 324 of FIG. 3.

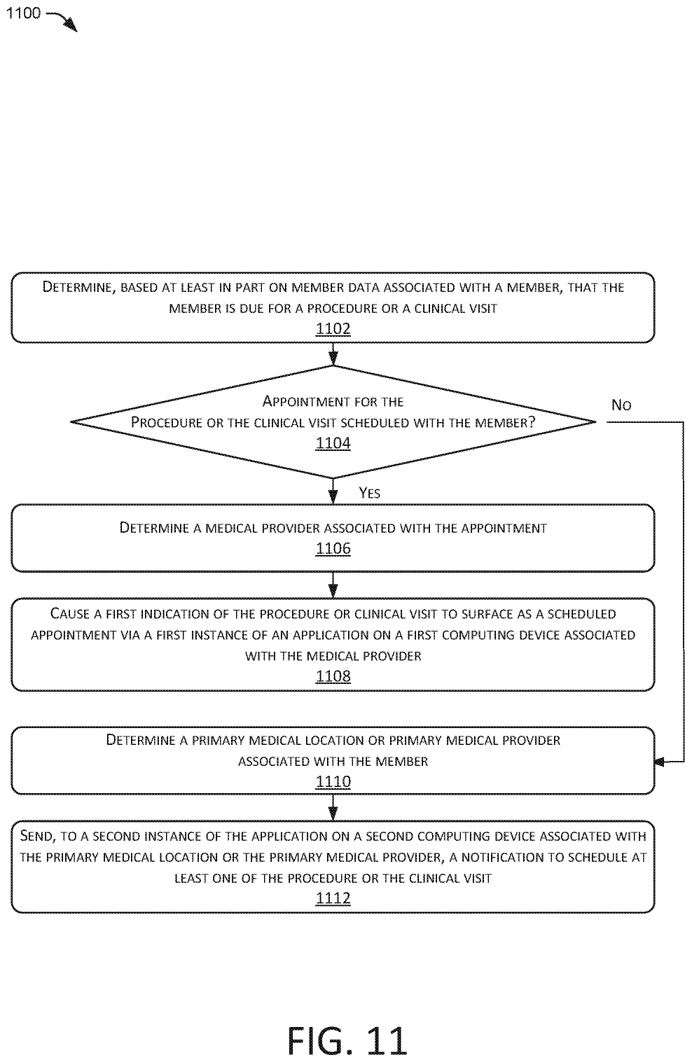

FIG. 11 illustrates an example process 1100 for determining that a member is due for a procedure and sending a notification of the procedure to a medical provider associated with the member, utilizing the techniques described herein. In some instances, some or all of process 1100 may be performed by one or more components in the systems 100 or 800. By way of example and not limitation, the service provider computing device (e.g., service provider) referred to in process 1100 may be representative of a computing device associated with the service provider 104 or service provider server(s) 802, the medical provider computing device (e.g., member device) referred to in process 1100 may be representative of the medical provider computing device(s) 106 and/or first computing device(s) 804 and the member computing device referred to in process 1100 may be representative of the member computing device(s) 110 and/or the second computing device(s) 806. However, the process 1100 is not limited to being performed by the system 100 or 800.

At operation 1102, the process 1100 may include determining, based at least in part on member data associated with a member, that the member is due for a procedure or a clinical visit. In various examples, a service computing device may determine that the member is due for the procedure or the clinical visit based on member data associated with the member. The member data may include last known clinical visit or procedure date, demographic information (e.g., age, race, ethnicity, gender, etc.), diagnoses associated with the member, health history, family health history, previous surgeries, and the like. In various examples, the service provider may determine that the member is due for the procedure or the clinical visit based on one or more clinical guidelines and/or member demographics. For example, a clinical guideline may include that women over the age of 50 have annual mammograms. Based on a determination that a time since a last mammogram associated with the member, a 53-year-old woman, is close to or more than a year, the service provider may determine that the member is due for the mammogram.

In some examples, the service provider may determine that a time since the last procedure or clinical visit is within a threshold time of the recommended time for the procedure. The threshold time may include any length of time determined by the service provider, such as 6 days, 14 days, or the like. In some examples, the threshold time may be based in part on the recommended time for the procedure or the clinical visit. For example, an annual procedure may have a longer threshold time than a quarterly procedure, or vice versa. In various examples, the threshold time may be determined based on an average time for the member and/or an average time associated with multiple members to schedule an appointment for the procedure or the clinical visit. In such examples, the service provider may access stored scheduling data associated with the member and/or a plurality of members to determine the average time. In some examples, the threshold time may be determined based on a medical provider preference, such as that stored in a medical provider account. For example, a particular medical provider may indicate to the service provider a preference to provide at least two months to schedule an appointment.

In some examples, the threshold time may be determined based on a level of importance (e.g., level of severity) associated with the procedure or clinical visit. In such examples, procedures or clinical visits with associated high levels of importance (e.g., critical to member health) may include a higher threshold time as compared to procedures or clinical visits with associated lower levels of importance (e.g., elective procedures). The level of importance or severity may be determined based on a one or more factors, such as member age, medical history, family history, statistical evaluation across a population, degree of life threat associated with illness corresponding to a procedure (e.g., colon cancer screening and life threat of colon cancer), and the like.

At operation 1104, the process 1100 may include determining whether an appointment for the procedure or clinical visit is scheduled with the member. In various examples, the service provider may access member data, a member schedule, and/or a medical provider schedule to determine whether the appointment for the procedure or clinical visit has been scheduled with the member. In some examples, the determination that the procedure or clinical visit has been scheduled may be based on a determination that a scheduling task (e.g., care task) to schedule the procedure or clinical visit has been completed.

Based on a determination that the appointment for the procedure or clinical visit has been scheduled (“Yes” at operation 1104), the process 1100 may include, at operation 1106, determining the medical provider associated with the appointment. In various examples, the service provider may determine the medical provider associated with the appointment based on an indication that an associate thereof completed a task associated with scheduling the appointment.

At operation 1108, the process 1100 may include causing a first indication of the procedure or clinical visit to surface or otherwise be presented as a scheduled appointment via a first instance of an application on a first computing device associated with the medical provider. In various examples, the first indication may surface or otherwise be presented as clinical visit (or procedure) task on a clinical visit (or procedure) page, such as the second status indicator 618(2) associated with the task 608(2) surfaced or presented on the clinical visit page 602 of FIG. 6.

Based on a determination that the appointment for the procedure or clinical visit has not been scheduled (“No” at operation 1104), the process 1100 may include, at operation 1110, determining a primary medical location or primary medical provider associated with the member. In some examples, the primary medical location or primary medical provider may be determined based on a determination of a primary care physician (or other provider) associated with the member. In such examples, the primary care physician may be stored in the member data associated with the member.

In some examples, the primary medical location or primary medical provider may be determined based on a procedure or clinical visit that is due for the member. In some examples, based on a determination that the procedure or clinical visit is associated with a specialty medical provider and/or medical location, the primary location and/or primary medical provider may include location and/or medical provider associated with the specialty. For example, based on a determination that the procedure is for a mammogram, the service provider may determine a primary medical location or primary medical provider associated with an imaging location and/or breast health clinic.

At operation 1112, the process 1100 may include sending, to a second instance of the application on a second computing device associated with the primary medical location or the primary medical provider, a notification to schedule at least one of the procedure or the clinical visit. In some examples, the notification may surface or otherwise be presented as a care task, such as care task 306 of FIG. 3. In some examples, the notification may surface or otherwise be presented as an electronic mail message, a short message system message, a message via the application, and/or a pop-up notification via the first instance of the application, such as notification 324 of FIG. 3.

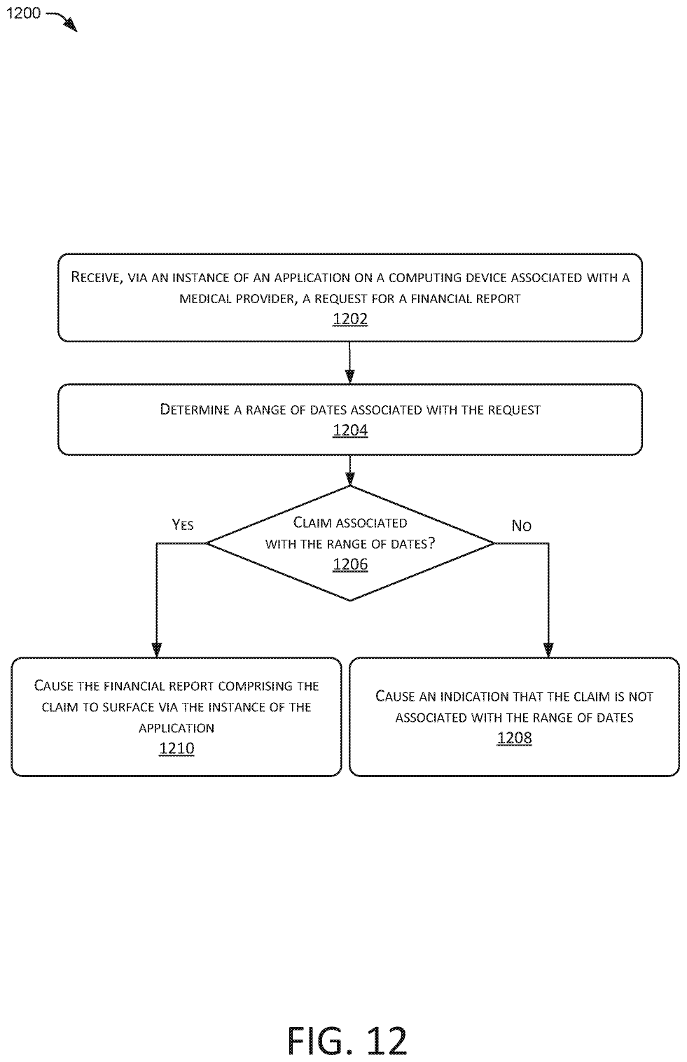

FIG. 12 illustrates an example process 1200 for providing a financial report to a medical provider. In some instances, some or all of process 1200 may be performed by one or more components in the systems 100 or 800. By way of example and not limitation, the service provider computing device (e.g., service provider) referred to in process 1200 may be representative of a computing device associated with the service provider 104 or service provider server(s) 802, the medical provider computing device (e.g., member device) referred to in process 1200 may be representative of the medical provider computing device(s) 106 and/or first computing device(s) 804 and the member computing device referred to in process 1200 may be representative of the member computing device(s) 110 and/or the second computing device(s) 806. However, the process 1200 is not limited to being performed by the system 100 or 800.

At operation 1202, the process 1200 may include receiving, via an instance of an application on a computing device associated with a medical provider, a request for a financial report. In some examples, the request for the financial report may include one or more filters for data, such as filters 720 of FIG. 7 including a date range (e.g., start date 722, end date 724), a date type 726 (e.g., type of service, date paid, etc.), a member 712, a provider 710, and/or the like. In various examples, the filters may provide a means by which a medical provider associate may specify data to be included in the financial report.

At operation 1204, the process 1200 may include determining a range of dates associated with the request. In some examples, the range of dates may be determined based on the start date and the end date, such as those input via the filters. In some examples, responsive to determining that a start date and end date were not included in the request, the service provider may determine the range of dates to include a time from the medical provider being associated with the service provider (e.g., a first date of service between the medical provider and a member associated with the service provider) and a date associated with the request.

At operation 1206, the process 1200 may include determining whether a claim is associated with the range of dates. In some examples, the determination regarding the claim may be based on a date type associated with the request. In such examples, the service provider may determine an association based on a date paid, a date of service, or the like. In some examples, the service provider may default to a date of service or a date paid, based on a determination that a date type is not associated with the request.

Based on a determination that the claim is not associated with the range of dates (“No” at operation 1206), the process 1200, at operation 1208, may include causing an indication that the claim was not associated with the range of dates.

Based on a determination that the claim is associated with the range of dates (“Yes” at operation 1206), the process 1200, at operation 1210, may include causing the financial report comprising the claim to surface or otherwise be presented via the instance of the application. The financial report may provide the medical associate with financial data associated with the range of dates and/or other filter data, such as provider information, member information, etc. The financial report may provide an indication of payments received for each claim and/or a total amount of payments received over the range of dates, such as for services rendered to members.

As stated above, the order in which the operations are described is not intended to be construed as a limitation, and any number of the described operations may be combined in any order and/or in parallel to implement the processes. In some embodiments, one or more operations of the above-described methods may be omitted entirely. By way of example and not limitation, operations 1002 and 1004 may be performed without operations 1006 and 1008 and/or operations 1102,1104, 1110 and 1112 may be performed without operations 1106-1108. Moreover, the methods described herein can be combined in whole or in part with each other or with other methods.

The various techniques described herein may be implemented in the context of computer-executable instructions or software, such as program modules, that are stored in computer-readable storage and executed by the processor(s) of one or more computing devices such as those illustrated in the figures. Generally, program modules include routines, programs, objects, components, data structures, etc., and define operating logic for performing particular tasks or implement particular abstract data types.

Other architectures may be used to implement the described functionality and are intended to be within the scope of this disclosure. Furthermore, although specific distributions of responsibilities are defined above for purposes of discussion, the various functions and responsibilities might be distributed and divided in different ways, depending on circumstances.

Similarly, software may be stored and distributed in various ways and using different means, and the particular software storage and execution configurations described above may be varied in many different ways. Thus, software implementing the techniques described above may be distributed on various types of computer-readable media, not limited to the forms of memory that are specifically described.

### CONCLUSION

Although the discussion above sets forth example implementations of the described techniques, other architectures may be used to implement the described functionality, and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A method comprising:
determining, based at least in part on member data associated with a member, that the member is due for at least one of a procedure or a clinical visit;
determining a medical provider associated with the member;
causing a scheduling task to be presented via a first instance of an application on a computing device associated with the medical provider, the scheduling task indicating for the at least one of the procedure or the clinical visit to be scheduled for the member;
receiving, via the first instance of an application on the computing device associated with a medical provider, a request to generate a clinical assessment associated with the clinical visit between the medical provider and the member;
determining, based at least in part on the member data, at least one of a potential diagnosis, a medication, a gap in care, or a clinical recommendation;
generating the clinical assessment comprising the at least one of the potential diagnosis, the medication, the gap in care, or the clinical recommendation; and
causing an indication of the clinical assessment to be presented via the first instance of the application on the computing device associated with the medical provider, the indication of the clinical assessment indicating that the clinical assessment associated with the clinical visit is generated.

2. The method of claim 1, further comprising:
receiving, via the first instance of the application, an input associated with the scheduling task, the input comprising at least one of:
a first date associated with a future appointment for the at least one of the procedure or the clinical visit; or
a second date associated with the procedure performed at a previous time; and
updating the member data based at least in part on the input.

3. The method of claim 1, further comprising:
determining, based at least in part on the member data, that the medication associated with the member is due for renewal within a threshold time;
generating a medication renewal task associated with the medication, the medication renewal task comprising medication data associated with the medication and a date associated with an expiration of the medication; and
causing the medication renewal task to be presented via the first instance of the application on the computing device associated with the medical provider.

4. The method of claim 3, further comprising:
receiving an input corresponding to the medication renewal task; and
updating the member data based at least in part on the input.

5. The method of claim 1, wherein determining that the member is due for the procedure comprises:
determining that the member is associated with the procedure based at least in part on at least one of:
an age associated with the member;
a diagnosis associated with the member;
a gender associated with the member;
a medical history associated with the member;
a family medical history associated with the member;
determining a periodic interval associated with the procedure, wherein the periodic interval is based at least in part on a clinical guideline associated with the procedure; and
determining, based at least in part on the member data, that at least one of:
the member has not previously undergone the procedure; or
a date associated with a previous procedure meets or exceeds the periodic interval associated with the procedure.

6. The method of claim 1, further comprising:
receiving, via the first instance of the application, a request for a financial report comprising a start date and an end date;
determining, based at least in part on the start date and the end date, one or more payments associated with the financial report; and
causing the one or more payments to be presented via the first instance of the application as the financial report.

7. The method of claim 1, further comprising:
receiving, via the first instance of the application, an indication of intent to schedule the at least one of the procedure or the clinical visit via the first instance of the application;
sending, to a member computing device associated with the member and via a second instance of the application, a notification to schedule the at least one of the procedure or the clinical visit;
receiving, from the member computing device associated with the member via the second instance of the application, an input comprising a date and time for an appointment associated with the at least one of the procedure or the clinical visit;
updating member data associated with the member based at least in part on the input comprising the date and the time for the appointment; and
causing a notification to be presented via the first instance of the application on the computing device associated with the medical provider, the notification indicating that the appointment is scheduled.

8. The method of claim 1, further comprising:
determining a first time associated with a previous appointment for the at least one of the procedure or the clinical visit;
determining a periodic interval associated with the at least one of the procedure or the clinical visit;
determining a second time based at least in part on the first time and the periodic interval, the second time being associated with the at least one of the procedure or the clinical visit; and
determining that a current time is within a threshold time of the second time,
wherein determining that the member is due for the at least one of the procedure or the clinical visit is based at least in part on determining that the current time is within the threshold time.

9. A system comprising:
one or more processors; and
computer-readable media storing instructions that, when executed by the one or more processors, cause the one or more processors to:
determine, based at least in part on member data associated with a member, that the member is due for at least one of a procedure or a clinical visit;
determine a medical provider associated with the member;
cause a scheduling task to be presented via a first instance of an application on a computing device associated with the medical provider, the scheduling task indicating for the at least one of the procedure or the clinical visit to be scheduled for the member;
receive, via the first instance of an application on the computing device associated with a medical provider, a request to generate a clinical assessment associated with the clinical visit between the medical provider and the member;
determine, based at least in part on the member data, at least one of a potential diagnosis, a medication, a gap in care, or a clinical recommendation;
generate a clinical assessment comprising the at least one of the potential diagnosis, the medication, the gap in care, or the clinical recommendation; and
cause an indication of the clinical assessment to be presented via the first instance of the application on the computing device associated with the medical provider, the indication of the clinical assessment indicating that the clinical assessment associated with the clinical visit is generated.

10. The system of claim 9, wherein the scheduling task is presented via the first instance of the application as a pop-up notification.

11. The system of claim 9, the instructions further causing the one or more processors to:
determine, based at least in part on the member data, that the medication associated with the member is due for renewal within a threshold time;
generate a medication renewal task associated with the medication, the medication renewal task comprising medication data associated with the medication and a date associated with an expiration of the medication; and
cause the medication renewal task to be presented via the first instance of the application on the computing device associated with the medical provider.

12. The system of claim 11, the instructions further causing the one or more processors to:
receive an input corresponding to the medication renewal task; and
update the member data based at least in part on the input.

13. The system of claim 9, the instructions further causing the one or more processors to:
determine a first time associated with a previous appointment for the at least one of the procedure or the clinical visit;
determine a periodic interval associated with the at least one of the procedure or the clinical visit;
determine a second time based at least in part on the first time and the periodic interval, the second time being associated with the at least one of the procedure or the clinical visit; and
determine that a current time is within a threshold time of the second time,
wherein determining that the member is due for the at least one of the procedure or the clinical visit is based at least in part on determining that the current time is within the threshold time.

14. The system of claim 13, wherein the threshold time is based at least in part on at least one of:
a clinical guideline associated with the at least one of the procedure or the clinical visit;
an age associated with the member;
a medical provider preference associated with the medical provider;
a member preference associated with the member;
a medical history associated with the member; or
a family medical history associated with the member.

15. One or more computer-readable media storing instructions that, when executed by one or more processors of a computing device, cause the computing device to:
determine, based at least in part on member data associated with a member, that the member is due for at least one of a procedure or a clinical visit;
determine a medical provider associated with the member;
cause a scheduling task to be presented via a first instance of an application on a computing device associated with the medical provider, the scheduling task indicating for the at least one of the procedure or the clinical visit to be scheduled for the member;
receive, via the first instance of an application on the computing device associated with the medical provider, a request to generate a clinical assessment associated with the clinical visit between the medical provider and the member;
determine, based at least in part on the member data, at least one of a potential diagnosis, a medication, a gap in care, or a clinical recommendation;
generate the clinical assessment comprising the at least one of the potential diagnosis, the medication, the gap in care, or the clinical recommendation; and
cause an indication of the clinical assessment to be presented via the first instance of the application on the computing device associated with the medical provider, the indication of the clinical assessment indicating that the clinical assessment associated with the clinical visit is generated.

16. The one or more computer-readable media of claim 15, the instructions further causing the computing device to:
determine medication data associated with the member, the medication data comprising one or more medications prescribed to the member; and
determine a potential modification to the medication of the one or more medications,
wherein determining that the member is due for the at least one of the procedure or the clinical visit is based at least in part on determining the potential modification to the medication.

17. The one or more computer-readable media of claim 15, the instructions further causing the computing device to:
determine, based at least in part on the member data, that the medication associated with the member is due for renewal within a threshold time;
generate a medication renewal task associated with the medication, the medication renewal task comprising medication data associated with the medication and a date associated with an expiration of the medication; and
cause the medication renewal task to be presented via the first instance of the application on the computing device associated with the medical provider.

18. The one or more computer-readable media of claim 15, the instructions further causing the computing device to:
receive, via the first instance of the application, a request for a financial report comprising a start date and an end date;
determine, based at least in part on the start date and the end date, one or more payments associated with the financial report; and
cause the one or more payments to be presented via the first instance of the application as the financial report.

19. The one or more computer-readable media of claim 15, the instructions further causing the computing device to:
determine that the member is associated with the procedure based at least in part on at least one of:
an age associated with the member;
a diagnosis associated with the member;
a gender associated with the member;
a medical history associated with the member; or
a family medical history associated with the member;
determine a periodic interval associated with the procedure, wherein the periodic interval is based at least in part on a clinical guideline associated with the procedure; and
determine, based at least in part on the member data, that at least one of:
the member has not previously undergone the procedure; or
a date associated with a previous procedure exceeds the periodic interval associated with the procedure.

20. The one or more computer-readable media of claim 15, the instructions further causing the computing device to:
determine a first time associated with a previous appointment for the at least one of the procedure or the clinical visit;
determine a periodic interval associated with the at least one of the procedure or the clinical visit;
determine a second time based at least in part on the first time and the periodic interval, the second time being associated with the at least one of the procedure or the clinical visit; and
determine that a current time is within a threshold time of the second time,
wherein determining that the member is due for the at least one of the procedure or the clinical visit is based at least in part on determining that the current time is within the threshold time.
